(19) United States
(12) Patent Application Publication
(10) Pub. No.: US 2011/0045564 A1
(43) Pub. Date: Feb. 24, 2011

(54) METHOD OF ENHANCED SUSTAINABLE PRODUCTION OF ALGAL BIO-PRODUCTS, COMPRISING USE OF SYMBIOTIC DIAZOTROPH-ATTENUATED STRESS CO-CULTIVATION

(76) Inventor: Srisuda Dhamwichukorn, Edmonds, WA (US)

Correspondence Address: DAVIS WRIGHT TREMAINE, LLP/Seattle, 1201 Third Avenue, Suite 2200, SEATTLE, WA, 98101-3045, US

(21) Appl. No.: 12/852,402

(22) Filed: Aug. 6, 2010

Related U.S. Application Data
(60) Provisional application No. 61/235,655, filed on Aug. 20, 2009.
(60) Provisional application No. 61/238,077, filed on Aug. 28, 2009.

(51) Int. Cl. C12P 1/04

(52) U.S. Cl. 435/170

(57) ABSTRACT

Provided are compositions and methods for sustainable cultivation of algae for biomass, biofuel and bioproduct production, preferably with minimal addition of exogenous nutrients, comprising co-cultivating at least one algal species with at least one aerobic bacterial species and at least one diazotroph (or, in certain embodiments, cultivation of at least one algal species with at least one diazotroph) under continuous sustainable symbiotic conditions, wherein a significant proportion of the macronutrients derive from endogenous decomposed algal and bacterial cells. Certain aspects provide continuous symbiotic diazotroph-attenuated nitrogen stress co-cultivation, wherein a continuous, balanced attenuated nitrogen-stress response provides for adequate sustained algal growth, while yet preserving advantages of algal nitrogen stress responses for algal bioproduct production. Preferred aspects provide for enhanced algal production of at least one of: lipids; triacylglycerols (TAGs); percentage of lips as TAGs; and percentage of saturated and mono-saturated fatty acids relative to polyunsaturated fatty acids (PUFAs) in TAGs.

### CROSS-REFERENCE TO RELATED APPLICATIONS

This application claims the benefit of priority to U.S. Provisional Patent Application Ser. Nos. 61/235,655, filed 20 Aug. 2009, entitled “Method for Enhanced Sustainable Production of Algal Bio-Products, Comprising Use of Symbiotic Diazotroph-Attenuated Stress Co-Cultivation,” and 61/238,077, filed 28 Aug. 2009, entitled “Apparatus and Method for Enhancing Disruption and Extraction of Intracellular Materials from Microbial Cells,” which are incorporated herein by reference in their entirety.

### FIELD OF THE INVENTION

Aspects of the invention relate generally to compositions and methods for sustainable cultivation of algae, and in particular aspects to compositions and methods for cultivation of a broad spectrum of algae for biomass production with minimal addition of exogenous nutrients, comprising co-culturing or co-cultivating at least one algal species with at least one aerobic bacterial species and at least one diazotroph (or at least one algal species with at least one diazotroph, in two requisite organismal component systems) under sustainable symbiotic conditions, and in preferred aspects wherein a significant proportion of the macronutrients for the symbiotic culture derive from decomposed algal and bacterial cells continuously produced during the symbiotic co-cultivation to provide a method for sustainable continuous culturing of algae with minimal addition of exogenous nutrients. Certain aspects relate to use of symbiotic diazotroph-attenuated nitrogen stress co-cultivation (DANSC). Preferred exemplary aspects relate to production of biofuels and other bioproducts using biomass produced by the disclosed compositions and methods.

### BACKGROUND

Algae are important resources for many beneficial bio-products. For example, algae cells contain pigments and other intracellular matters for nutraceuticals, vitamins, bioplastics, dyes and colorants, feedstocks, pharmaceuticals, algae fuels and especially oils for energy and health care purposes. Algal cells contain proteins, carbohydrates and fatty acids or oil. Proteins can be used as protein supplement or feedstock. Carbohydrates can be used for biogas and bioethanol production. Oil and fatty acids can be used as biocrude or oil for biodiesel production. In addition, pigments, oils or many intracellular materials can be used for pharmaceuticals or nutraceuticals.

The cultivation of algae, similar to culturing many other microorganisms, requires both macro and micro nutrients. Both macro and micronutrients can be obtained from either organic or inorganic sources. However, obtaining nutrients from organic sources is safer and healthier than from synthetic chemicals. In addition, the cultivation using organic nutrients is more environmentally friendly. With these advantages, organic cultivation of algae provides added value to the algal products and thus higher benefits for investment, especially if macronutrients as major portion of nutrients are from organic sources.

Many examples of algal cultivation exist in the art. In particular, examples of closed photobioreactors to culture algae include U.S. Pat. Nos. 2,732,663; 4,473,970; 4,233,958; 4,868,123; and 6,827,036. More recently, Pulz and Scheibenbogen (Pulz O. and Scheibenbogen K. “Photobioreactors: Design and Performance with Respect to Light Energy Input,” Advances in Biochemical Engineering/Biotechnology, 59:pp 124-151 (1998); hereinafter “Pulz 1998”) reviewed algae photobioreactors, and Richmond (Richmond A. ed.) “Handbook of Microalgal Culture—Biotechnology and Applied Phycology”, Blackwell Publishing, Oxford, UK (2004); hereinafter “Richmond 2004”) reviews the general state of the art of microalgae culturing, including reactor design. Both references (Richmond 2004 and Pulz 1998) note that open systems (e.g.,-raceway reactors) are the predominant commercial technology. Open air systems used for cultivation of algae are also shown in, for example, U.S. Pat. Nos. 3,650,068; 3,468,057; 3,955,318; and 4,217,728.

Typical prior art commercial algal growth methods, however, rely on the use of exogenously added fertilizers and chemicals for the bulk of the macro and micronutrients needed to sustain the algal cultures, and are, therefore, not only energy intensive and expensive, but are also environmentally hostile. Additionally, most commercial bioreactors have been optimized and are suitable for only a limited number of algal species.

There is, therefore, from both environmental and economic perspectives a pronounced need in the art for novel compositions and methods to provide for sustained growth of a broad variety of algae with less reliance on exogenously added fertilizers and chemicals to sustain the algal cultures (e.g., using surface water and/or groundwater as the primary culture medium).

### SUMMARY OF THE INVENTION

Provided are compositions and methods for sustainable cultivation of algae for biomass, biofuel and bioproduct production, preferably with minimal addition of exogenous nutrients, comprising co-cultivating at least one algal species with at least one aerobic bacterial species and at least one diazotroph (or, in certain embodiments, cultivation of at least one algal species with at least one diazotroph) under continuous sustainable symbiotic conditions, wherein a significant proportion of the macronutrients derives from endogenous decomposed algal and bacterial cells. Certain aspects provide continuous symbiotic diazotroph-attenuated nitrogen stress co-cultivation (DANSC), wherein a continuous, balanced attenuated nitrogen-stress response provides for adequate sustained algal growth, while yet preserving advantages of algal nitrogen stress responses for algal bioproduct production. Preferred aspects provide for enhanced algal production of at least one of: lipids; triacylglycerols (TAGs); percentage of lips as TAGs; and percentage of saturated and mono-saturated fatty acids relative to polyunsaturated fatty acids (PUFAs) in TAGs. The methods are broadly applicable to many types of algae, and can be practiced with a broad range of suitable aerobic bacterial symbiotic organisms, and suitable diazotrophic symbiotic organisms.

In particular preferred aspects, maintaining a balanced symbiotic co-culture as described herein not only enables algal growth using low exogenous nutrient growth addition, but enables algal growth with a diazotroph-attenuated, stress-enhanced bioproduct (e.g., lipid, oil, TAG) yield (e.g., on a per-algal cell basis) using low exogenous nutrient growth addition. Applicant refers to this herein as symbiotic diazotroph-attenuated nitrogen stress co-cultivation (DANSC). While nitrogen stress responses in algae are known in the art, prior art attempts at using nitrogen stressed to induce algal bioproduct production have been limited to closed-system bioreactors where algae are initially non-symbiotically grown in rich chemical medium to provide a large algal biomass, followed by imposing nitrogen deprivation by rapid exhaustion and/or adjustment of nutrients in the medium of the closed system to induce nitrogen stress responses, followed by complete batch harvesting of the nitrogen stress algal biomass; that is, prior art methods comprise non-continuous batch processes that are suitable for closed systems only. By contrast, Applicant's inventive methods comprise the use of continuous symbiotic diazotroph-attenuated nitrogen stress co-cultivation (DANSC), as disclosed and taught herein, to provide for a continuous co-culture using diazotroph-attenuated nitrogen stress such that the advantages of nitrogen stress for algal bioproduct production can be implemented and sustained continuously in batch or non-batch processes, and in open and/or closed cultivation systems. Applicant's disclosed advantageous use of diazotrophs in the context of nitrogen-stressed algae is not only novel, but is counterintuitive and unexpected, because provision of bioavailable nitrogen to the algal co-cultures by addition of diazotrophs would not only be expected to decrease any advantages of nitrogen stress for algal bioproduct production, but would also be expected to cause nutrient depletion by the diazotrophs thereby limiting algal growth in the co-cultures. However, the Applicant has surprisingly discovered that symbiotic growth in the inventive co-cultures with diazotrophs provides for adequate sustained algal growth, while yet adequately preserving the advantages of nitrogen stress for algal bioproduct production by providing a balanced attenuated nitrogen stress response in the continuous co-culture. Applicant's methods, therefore, provide commercially adequate biomass yield with a nitrogen-stress-enhanced bioproduct content, which, unlike prior art nitrogen stress batch processes, can be sustained on a continuous symbiotic basis in open or closed systems.

Particular aspects provide methods for enhanced sustainable production of algal bioproducts, comprising: providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel; inoculating the cultivation medium in the vessel with at least one algal species, at least one aerobic bacterial species and at least one diazotroph; continuously cultivating the inocula under sustainable symbiotic co-culture conditions to provide for diazotroph-assisted sustained production of a harvestable amount of algal biomass; and repetitive harvesting of a portion of the algal biomass from the continuous co-culture, to provide for enhanced sustainable production of at least one algal bioproduct.

Additional aspects provide methods for enhanced sustainable production of algal bioproducts, comprising: providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel, the cultivation medium suitable to induce at least one nitrogen stress response in algal cells cultured therein; inoculating the cultivation medium in the vessel with at least one algal species, at least one aerobic bacterial species and at least one diazotroph; continuously cultivating the inocula under sustainable symbiotic co-culture conditions, wherein the diazotroph component is maintained in an amount sufficient to sustainably attenuate the at least one nitrogen stress response in the symbiotically co-cultivated algal cells to provide for diazotroph-assisted sustained production of a harvestable amount of algal biomass; and repetitive harvesting of a portion of the algal biomass from the continuous co-culture, to provide for enhanced sustainable production of at least one algal bioproduct.

Further aspects provide methods for enhanced sustainable production of algal bioproducts, comprising: providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel, the cultivation medium suitable to induce at least one nitrogen stress response in algal cells cultured therein; inoculating the cultivation medium in the vessel with at least one algal species, at least one aerobic bacterial species and at least one diazotroph; continuously cultivating the inocula under sustainable symbiotic co-culture conditions, wherein at least a portion of the algal growth in the co-culture is photosynthetic, and wherein the diazotroph component is maintained in an amount sufficient to sustainably attenuate the at least one nitrogen stress response in the symbiotically co-cultivated algal cells to provide for diazotroph-assisted sustained production of a harvestable amount of algal biomass; and repetitive harvesting of a portion of the algal biomass from the continuous co-culture, to provide for enhanced sustainable production of at least one algal bioproduct.

Yet further aspects provide a method for enhanced sustainable production of algal bioproducts, comprising: providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel; inoculating the cultivation medium in the vessel with at least one algal species, and at least one diazotroph; continuously cultivating the inocula under sustainable symbiotic co-culture conditions to provide for diazotroph-assisted sustained production of a harvestable amount of algal biomass; and repetitive harvesting of a portion of the algal biomass from the continuous co-culture, to provide for enhanced sustainable production of at least one algal bioproduct.

In certain aspects of the above-summarized methods, at least a portion of the algal growth in the co-culture is photosynthetic, and/or algal growth comprises both heterotrophic and autotrophic growth.

In particular three requisite organismal component embodiments of the methods, inoculating comprises use of an initial inoculum ratio of algae:aerobic bacteria:diazotroph selected from the group consisting of: 100:1.6:0.18; 10:1.6:18; 50-500:0.8-80:0.09-9; and 10-1000:0.16-160:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:1.6:0.18; 100:25:18; 50-500:0.8-80:0.09-9; and 10-1000:0.16-160:0.018-18, or comprises a sustained symbiotic ratio of algae:aerobic bacteria:diazotroph, including dead biomass, selected from the group consisting of: 110:10:1.5; 150:50:15; 55-550:5-50:0.75-7.5; and 15-1100:1-100:0.15-15.

In particular two requisite organismal component embodiments of the methods, inoculating comprises use of an initial inoculum ratio of algae:diazotroph selected from the group consisting of: 100:0.18; 10:18; 50-500:0.09-9; and 10-1000:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:0.18; 100:18; 50-500:0.09-9; and 10-1000:0.018-18, or comprises a sustained symbiotic ratio of algae:aerobic bacteria:diazotroph, including dead biomass, selected from the group consisting of: 110:1.5; 150:15; 55-550:0.75-7.5; and 15-1100:0.15-15.

In certain aspects of the methods, the methods comprise or further comprise monitoring of the at least one of CO₂, O₂, nitrogen, and pH levels in the cultivation medium; and adjusting the at least one of CO₂, O₂, nitrogen, and pH levels in the cultivation medium as required to provide for sustainable symbiotic co-culture of the at least one algal species, the at least one aerobic bacterial species and the at least one diazotroph, or, in the case of two requisite organismal component embodiments, of the at least one algal species and the at least one diazotroph.

In particular aspects of the methods, the methods comprise or further comprise isolating at least one algal bioproduct from the harvested algal biomass (e.g., biofuel, biocrude, bioenergy, biogas, biodiesel, bioethanol, biogasoline, biocrude, pharmaceuticals, therapeutics, antioxidants, nutraceuticals, cosmetics, cosmeceuticals, food, feedstock, dyes, colorants, bioplastics, etc.).

In preferred embodiments of the methods, sustainable growth of the at least one algal species, the at least one aerobic bacterial species and the at least one diazotroph, or of the at least one algal species and the at least one diazotroph in two component systems, is maintained with low nutrient addition. In particular preferred aspects, the methods comprise use of minimal addition of exogenous nutrients, and preferably wherein at least 5% of the macronutrient driving growth in the symbiotic co-culture derive from decomposed algal and bacterial cells produced during the co-cultivating.

In preferred embodiments of the methods, the cultivation medium is suitable to induce at least one nitrogen stress response in the algal cells cultured therein, and in particularly preferred embodiments, the diazotroph component is maintained in an amount sufficient to sustainably attenuate the at least one nitrogen stress response in the symbiotically co-cultivated algal cells. In particular aspects of the methods, the aqueous cultivation medium comprises at least one of ground water, surface water, brackish water, salt water, sea water, marine water, lake water, river water, waste water, and tap water.

In preferred embodiments of the methods, at least a portion of the CO₂ present in the cultivation medium is endogenously derived from the aerobic bacterial component of the co-culture, at least a portion of the nitrogen present in the cultivation medium is endogenously derived from the diazotrophic component of the co-culture, and at least a portion of the O₂ present in the cultivation medium is endogenously derived from the algal component of the co-culture. In preferred embodiments of two requisite organismal component embodiments of the methods, at least a portion of the nitrogen present in the cultivation medium is endogenously derived from the diazotrophic component of the co-culture, and at least a portion of the O₂ present in the cultivation medium is endogenously derived from the algal component of the co-culture.

In particular embodiments of the methods, the co-culture provides, on a per-algal cell basis, relative to non-symbiotic growth of the respective algal cells, for at least one of: enhanced total lipid production; enhanced production of triacylglycerols (TAGs); enhanced percentage of total lipid as TAGs; and enhanced percentage of saturated and mono-saturated fatty acids, relative to polyunsaturated fatty acids (PUFAs), in TAGs. In certain aspects of the methods, the total lipid content is enhanced to a level equal to or greater than: 30%; 35%; 40%; 45%; or 50% dry cell weight (DCW), or enhanced to a value in the range of from about 30% to about 50% DCW. In certain embodiments of the methods, the amount of total lipid in the form of triacylglycerols (TAGs) is equal to or greater than: 20%; 30%; 40%; 50%; 60%; 70%; or 80% dry cell weight (DCW)of the total lipid, or in the range of from about 30% to about 80% DCW of the total lipid. In particular aspects of the methods, the increased percentage, relative to polyunsaturated fatty acids (PUFAs), of the saturated and mono-saturated fatty acids in the triacylglycerols (TAGs), is at least: 5%; 10%; 20%; 30% dry cell weight (DCW); or greater, or is in the range of from about 10% to about 30% DCW.

In particular embodiments of the methods, the at least one diazotroph is selective from the diazotrophic bacterial group consisting of photosynthetic, non-photosynthetic, anaerobic, aerobic, methanogenic, sulfurgenic, symbiotic diazotrophes, cyanobacteria, and oxygenic and anoxygenic forms thereof. In certain embodiments of the methods, the at least one algal species, and/or the at least one aerobic bacterial species, and/or the at least one diazotroph comprises at least one organism according to Tables 1-4 as disclosed herein.

### BRIEF DESCRIPTION OF THE DRAWINGS

FIG. 1 shows, according to particular exemplary aspects, the conceptual basis underlying Applicant's sustained co-culture and life cycle-enhanced cultivation of algae.

FIG. 2 summarizes, according to particular exemplary aspects, the operation of sustained co-culture and life cycle-enhanced cultivation of algae.

FIG. 3 demonstrates, according to particular aspects, two exemplary scenarios occurring in the system. Scenario A applies when there is sufficient photosynthesis to keep an appropriate balance of oxygen and carbon dioxide for algae growth. Scenario B applies when there is no photosynthesis or insufficient photosynthesis (B1) and aeration, for example, is needed to supply sufficient oxygen and/or purge carbon dioxide (B2).

FIG. 4 shows, according to particular exemplary aspects, an automated cultivation system for producing algae to be processed for bioproducts and bioenergy.

FIG. 5 shows, according to particular exemplary aspects, a manually controlled cultivation system for producing algae to be processed for bioproducts and bioenergy.

FIG. 6 displays, according to particular exemplary aspects, a variety of exemplary beneficial bioproducts that can be produced from algae obtained from the disclosed symbiotic co-cultivation systems.

### DETAILED DESCRIPTION OF THE INVENTION

Particular aspects provide compositions and methods for sustainable cultivation of algae, and in particular aspects provide compositions and methods for cultivation of a broad spectrum of algae for biomass production with minimal addition of exogenous nutrients, comprising co-culturing or co-cultivating at least one algal species with at least one aerobic bacterial species and at least one diazotroph under sustainable symbiotic conditions, wherein a significant proportion of the macronutrients for the symbiotic culture derive from decomposed algal and bacterial cells continuously produced during the symbiotic co-cultivation to provide a method for sustainable continuous culturing of algae with minimal addition of exogenous nutrients. Preferred exemplary aspects provide for production of biofuels and other bioproducts using biomass produced by the disclosed compositions and methods. The methods are broadly applicable to many types of algae, and can be practiced with a broad range of suitable aerobic bacterial symbiots and suitable diazotrophic organisms.

Certain aspects of the invention are directed to symbiotic co-cultivation of at least one algae with at least one aerobic bacterial species as described herein.

Certain embodiments and aspects of the present invention relate to sustainable, symbiotic co-cultivation of at least one algae species, at least one aerobic bacterial species, and at least one diazotropic organism in a suitable vessel/container (e.g., photobioreactor apparatus, open pond, or raceway pond) designed to contain a liquid medium. Table 1 shows exemplary preferred organisms for use in synergistic combinations of algae, aerobic bacteria, and diazotrophs.

According to certain embodiments, the source of a significant proportion of the carbon, nitrogen, phosphorus, potassium, and other macro nutrients for the living organisms of the symbiotic co-culture comes from disrupted or dead algal and microbial (e.g., bacterial) cells that are continuously produced during the co-cultivation.

Certain aspects of the invention are directed to symbiotic co-cultivation of algae, or of algae and an aerobic bacterial species, with a diazotrophic organism. Diazotrophic organisms, as used herein, are organisms that fix elemental nitrogen into a more usable form such as ammonia. A review of nitrogen-fixing organisms is provided by Postgate, J (1998) Nitrogen Fixation, 3rd Editio. Cambridge University Press, Cambridge UK. In preferred embodiments, the diazotropic organism may include but is not limited to at least one of the diazotrophic organisms listed in Tables 3 and 4. Suitable diazotrophs can be found in almost all bacterial taxonomic groups. One phylum, in particular, which includes a large number of suitable nitrogen-fixing bacteria is cyanophyta. Table 3, for example, lists exemplary genera and species of cyanobacteria encompassed by the present invention. Additional suitable genera and species of exemplary diazotrophic bacteria are listed in Table 4, and are additionally encompassed by the present invention.

According to preferred embodiments, as indicated in Table 1, at least one type/class of algae can be used in combination with at least one type/class of aerobic bacteria, and further in combination with at least one type of diazotrophic bacteria, and/or photosynthetic diazotrophic bacteria (e.g., at least one cyanobacteria) to fix nitrogen. Tables 2, 3, and 4 show a representative number of exemplary species for each genus of algae, aerobic bacteria, and diazotoph (e.g., photosynthetic, nitrogen-fixing bacteria, such as cyanobacteria), that are suitable for use in the methods of the invention.

Certain aspects of the invention are directed to co-cultivation with an algae species. In preferred embodiments the algae species include but are not limited to marine, brackish water and freshwater algae. In further aspects, the algae species include but are not limited to those species that are derived from acidic or basic water. According to particular aspects, the algae species include any micro or macro algal species, including but not limited to, any eukaryotic algae such as diatoms and green, red, and brown algae e.g., kelp. In particular aspects of the invention the algae species includes but is not limited to those phyla, genera, and species listed in Table 2.

Exemplary algal species include, but are not limited to, Chlorella vulgaris, Chlorella ellipsoidea, Chlorella pyrenoidosa, Chlorella spp, Scenedesmus Acuminatus, Scenedesmus obliquus, Scenedesmus quadricauda, Scenedesmus dimorphus, Scenedesmus spp., Chlamydomonas rheinhardii, Chlamydomonas globosa, Chlamydomonas angulosa, Chlamydomonas spp., Spirogyra neglecta, Spirogyra gracilis, Spirogyra spp, Euglena rostrifera, Euglena gracilis, Euglena spiroides, Euglena anabaena, Euglena spp., Navicula cancellata, Navicula menisculus, Navicula perminuta, Navicula spp., Aulacoseira islandrica, Aulacoseira muszanensis, Aulacoseira alpigena, Aulacoseira spp., Microspora floccosa, Microspora spp., Batrachospermum turfosum Batrachospermum gelatinosum, Batrachospermum spp., Compsopogonopsis fruticosa, Compsopogon minutes, Compsogogon spp., Audouinella glomerata, Audouinella cylindrical, and Audouinella spp.

Exemplary aerobic bacterial species include, but are not limited to those from the classes of Gammaproteobacteria (e.g., Escherichia, Pseudomonas), Actinobacteria (e.g., Rhodococcus), Bacilli, (e.g., Bacillus), Beta Proteobacteria (e.g., Achromobacter) and Alphaproteobacteria (e.g., Rhodobacter).

Exemplary diazotrophic species include, but are not limited to, Anabaena Siamensis, Anabaena spiroides, Anabaena cylindrical, Anabaena spp, Spirulina Platensis, Spirulina maxima, Spirulina spp, Calothrix marchica, Calothrix spp., Lyngbya perelegans, Lyngbya wollei, Lyngbya spp., Hapalosiphon Hybernicus, Hapalosiphon spp., Nostoc linckia, Nostoc commune, Nostoc spp., Oscillatoria borneti, Oscillatoria limosa, Oscillatoria princeps, Oscillatoria salina, Oscillatoria okeni, Oscillatoria spp, Gloeocapsa gelatinosa, Gloeocapsa spp., Microcoleu Chthonoplates, Microcoleus spp., Aphanothece stagnina, Aphanothece clathrata, Aphanothece granulosa, Aphanothece spp., Klebsiella pneumonia, Klebsiella spp., Bacillus polymyxa, Bacillus macerans, Bacillus spp., Escherichia intermedia, Escherichia spp., Paenibacillus polymyxa, Paenibacillus macerans, Paenibacillus spp., Azobacter vinelandii, Azobacter spp., Rhodobacter sphaeroides, Rhodobacter capsulatus, Rhodobacter spp., Rhodopseudomonas palustris, Rhodopseudomonas spp., Methanosarcina barkeri, Methanosarcina spp., Methanospirillum hungateii, Methanospirillum spp., Methanobacterium bryantii, and Methanobacterium spp.

TABLE 1
Exemplary Preferred Synergistic Combinations
of Algae, Aerobic Bacteria, and Diazotrophs.

| | | Diazotrophs | |
|---|---|---|---|
| | | | Additional Nitrogen |
| Algal Phyla and | Aerobic Bacterial | Nitrogen Fixing | Fixing Bacteria and |
| Genera | Classes | Cyanobacteria | Archaea |
| Chlorophyta: | Gammaproteobacteria | Anabaena, | Klebsiella, |
| Chlorella, | (e.g. Escherichia, | Nostoc, | Bacillus, |
| Scenedesmus, | Pseudomonas) | Spirulina, | Escherichia, |
| Chlamydomonas, | Actinobacteria (e.g. | Synechococcus, | Paenibacillus, |
| Closterium, | Rhodococcus) | Oscillatoria, | Azobacter, |
| Synedra, | Bacillus | Synechocystis, | Rhodobacter, |
| Pediastrum, | Clostridium | Gloeocapsa, | Rhodopseudomonas, |
| Ankistrodesmus, | Beta Proteobacteria (e.g. | Hapalosiphon, | Methanosarcina, |
| Planktosphaeria, | Achromobacter) | Stigonema, | Methanospirillum, |
| Mougeotia | | Microcoleus, | Methanobacterium |
| | | Aphanothece | |
| Euglenophyta: | Gammaproteobacteria | Anabaena, | Klebsiella, |
| Euglena, | (e.g. Escherichia, | Nostoc, | Bacillus, |
| | Pseudomonas) | Spirulina, | Escherichia, |
| | Actinobacteria(e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria(e.g. | Hapalosiphon, | Methanosarcina, |
| | Achromobacter) | Stigonema, | Methanospirillum, |
| | | Microcoleus, | Methanobacterium |
| | | Aphanothece | |
| Bacillariophyta: | Gammaproteobacteria | Anabaena, | Klebsiella, |
| Navicula, | (e.g. Escherichia, | Nostoc, | Bacillus, |
| Surirella | Pseudomonas) | Spirulina, | Escherichia, |
| | Actinobacteria(e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria(e.g. | Hapalosiphon, | Methanosarcina, |
| | Achromobacter) | Stigonema, | Methanospirillum, |
| | | Microcoleus, | Methanobacterium |
| | | Aphanothece | |
| Microspora: | Gammaproteobacteria | Anabaena, | Klebsiella, |
| Microspora | (e.g. Escherichia, | Nostoc, | Bacillus, |
| | Pseudomonas) | Spirulina, | Escherichia, |
| | Actinobacteria(e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria(e.g. | Hapalosiphon, | Methanosarcina, |
| | Achromobacter) | Stigonema, | Methanospirillum, |
| | | Microcoleus, | Methanobacterium |
| | | Aphanothece | |
| Xanthophyta: | Gammaproteobacteria | Anabaena, | Klebsiella, |
| Tribonemu, | (e.g. Escherichia, | Nostoc, | Bacillus, |
| | Pseudomonas) | Spirulina, | Escherichia, |
| | Actinobacteria(e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria(e.g. | Hapalosiphon, | Methanosarcina, |
| | Achromobacter) | Stigonema, | Methanospirillum, |
| | | Microcoleus, | Methanobacterium |
| | | Aphanothece | |
| Rhodophyta: | Gammaproteobacteria | Anabaena, | Klebsiella, |
| Compsopogonopsis, | (e.g. Escherichia, | Nostoc, | Bacillus, |
| Audoouinella | Pseudomonas) | Spirulina, | Escherichia, |
| | Actinobacteria(e.g. | Synechococcus, | Paenibacillus, |
| | Rhodococcus) | Oscillatoria, | Azobacter, |
| | Bacillus | Synechocystis, | Rhodobacter, |
| | Clostridium | Gloeocapsa, | Rhodopseudomonas, |
| | Beta Proteobacteria(e.g. | Hapalosiphon, | Methanosarcina, |
| | Achromobacter) | Stigonema, | Methanospirillum, |
| | | Microcoleus, | Methanobacterium |
| | | Aphanothece | |

TABLE 2
Exemplar Genera of Algae in the Phyla and their Exemplar
Species including Identified and Unidentified Species.

| Phyla | Genera | Exemplar Species |
|---|---|---|
| Chlorophyta | Chlorella | Chlorella vulgaris, Chlorella |
| | | ellipsoidea, Chlorella |
| | | pyrenoidosa, Chlorella spp |
| | Scenedesmus | Scenedesmus Acuminatus, |
| | | Scenedesmus obliquus, |
| | | Scenedesmus quadricauda, |
| | | Scenedesmus dimorphus, |
| | | Scenedesmus spp. |
| | Chlamydomonas | Chlamydomonas rheinhardii, |
| | | Chlamydomonas globosa, |
| | | Chlamydomonas angulosa, |
| | | Chlamydomonas spp. |
| | Spirogyra | Spirogyra neglecta, Spirogyra |
| | | gracilis, Spirogyra spp. |
| Euglenophyta | Euglena: | Euglena rostrifera, Euglena |
| | | gracilis, Euglena spiroides, |
| | | Euglena anabaena, Euglena spp. |
| Bacillariophyta | Navicula | Navicula cancellata, Navicula |
| | | menisculus, Navicula |
| | | perminuta, Navicula spp. |
| | Aulacoseira | Aulacoseira islandrica, |
| | | Aulacoseira muszanensis, |
| | | Aulacoseira alpigena, |
| | | Aulacoseira spp. |
| Microspora | Microspora | Microspora floccosa, |
| | | Microspora spp. |
| Rhodophyta | Batrachospermum | Batrachospermum turfosum |
| | | Batrachospermum |
| | | gelatinosum, |
| | | Batrachospermum spp. |
| | Compsopogonopsis | Compsopogonopsis fruticosa, |
| | | Compsopogon minutes, |
| | | Compsogogon spp. |
| | Audoouinella | Audouinella glomerata, |
| | | Audouinella cylindrical, |
| | | Audouinella spp. |

TABLE 3
Exemplar Cyanobacteria and their Exemplar Species

| Genera | Exemplar Species |
|---|---|
| Anabaena | Anabaena Siamensis, Anabaena spiroides, |
| | Anabaena cylindrical, Anabaena spp. |
| Spirulina | Spirulina Platensis, Spirulina maxima, Spirulina spp. |
| Calothrix | Calothrix marchica, Calothrix spp. |
| Lyngbya | Lyngbya perelegans, Lyngbya wollei, Lyngbya spp. |
| Hapalosiphon | Hapalosiphon Hybernicus, Hapalosiphon spp. |
| Nostoc | Nostoc linckia, Nostoc commune, Nostoc spp. |
| Oscillatoria | Oscillatoria borneti, Oscillatoria limosa, |
| | Oscillatoria princeps, Oscillatoria salina, |
| | Oscillatoria okeni, Oscillatoria spp. |
| Gloeocapsa | Gloeocapsa gelatinosa, Gloeocapsa spp. |
| Microcoleus | Microcoleu Chthonoplates, Microcoleus spp. |
| Aphanothece | Aphanothece stagnina, Aphanothece clathrata, |
| | Aphanothece granulosa, Aphanothece spp. |

TABLE 4
Exemplary Additional Nitrogen-fixing Bacteria
and Archaea and their Exemplar Species.

| Genera | Exemplar Species |
|---|---|
| Klebsiella | Klebsiella pneumonia, Klebsiella spp. |
| Bacillus | Bacillus polymyxa, Bacillus macerans, Bacillus spp. |
| Escherichia | Escherichia intermedia, Escherichia spp. |
| Paenibacillus | Paenibacillus polymyxa, Paenibacillus macerans, |
| | Paenibacillus spp. |
| Azobacter | Azobacter vinelandii, Azobacter spp. |
| Rhodobacter | Rhodobacter sphaeroides, Rhodobacter capsulatus, |
| | Rhodobacter spp. |
| Rhodopseudomonas | Rhodopseudomonas palustris, |
| | Rhodopseudomonas spp. |
| Methanosarcina | Methanosarcina barkeri, Methanosarcina spp. |
| Methanospirillum | Methanospirillum hungateii, Methanospirillum spp. |
| Methanobacterium | Methanobacterium bryantii, Methanobacterium spp. |

Certain aspects of the invention are directed to symbiotic co-cultivation of algae for the production of biofuel, biocrude or bioenergy, including but not limited to biogas, biodiesel, bioethanol, biogasoline, biocrude, pharmaceuticals, therapeutics, antioxidants, nutraceuticals, cosmetics, cosmeceuticals, food, feedstock, dyes, colorants and bioplastic, depending on the algal species and metabolic conditions being used in the symbiotic co-cultivation systems.

According to certain embodiments, certain types of bacteria provide for specific desirable conditions when used in symbiotic co-culture or co-cultivation with algae. For example, aerobic bacteria can be used to provide CO₂ for algal growth. Alternatively, or additionally, cyanobacteria can be used provide for nitrogen fixing in symbiotic co-cultures, wherein the goal as disclosed herein is to enable growth of algae using low exogenous nutrient growth addition. According to particular aspects, adding in at least one nitrogen-fixing organism (e.g., at least one nitrogen fixing bacteria and/or photosynthetic, nitrogen-fixing bacteria, such as at least one cyanobacteria) to fix nitrogen provides for a substantial reduction in the requirement for exogenous nitrogen.

As recognized in the art (e.g., Hu, et al., The Plant Journal, 54:621-639, 2008; Alonso et al., Phytochemistry 54:461-471, 2000; Renaud et al., Aquaculture 211:195-214, 2002, all incorporated herein by reference, and in particular for their teachings one oil content and lipid and fatty acid compositions), stress of algae, and particularly based on nitrogen deprivation enhances (e.g., on a per cell basis) oil production by the stressed algae. According to additional aspects, the inventive symbiotic co-cultures comprising a diazotroph provide for enhanced oil production (e.g., sustained enhanced oil production) by the algae compared to oil production by non-nitrogen-stressed algae in cultures lacking a diazotroph. Without being bound by mechanism, the disclosed inventive use of a diazotroph in the inventive symbiotic co-cultures, in the absence of exogenously added chemical nitrogen, not only provides bioavailable nitrogen, but also unexpectedly provides for enhanced oil production by the algal component (e.g., on a per cell basis) of the symbiotic co-culture by providing an amount of bio-available nitrogen that is, on the one hand, sufficient to provide for healthy algal growth within the co-culture without, on the other hand, abrogating the art-recognized nitrogen-stress-mediated enhancement of oil production by the algae. Therefore, according to preferred aspects, maintaining a balanced symbiotic co-culture as described herein not only enables algal growth using low exogenous nutrient growth addition, but enables algal growth with an enhanced oil yield (e.g., on a per-cell basis) using low exogenous nutrient growth addition (see Example 7 herein below for further discussion of this aspect).

According to certain embodiments, algae growth within the system is heterotrophic, as defined herein. According to further embodiments, at least a certain percentage of the algae is growing heterotrophically (e.g, instead of autotrophically), for example, at least: 5%; 10%; 15%; 20%; 25%; 30%; 35%; 40%; 45%; 50%; 55%; 60%; 65%; 70%; 75%; 80%; 85%; 90%; or 95% In preferred embodiments, the percentage of heterotrophic growth is at least: 15%; 20%; 25%; 30%; 35%; 40%; 45%; or 50%. Said heterotrophic growth may be continuous, intermittent, cyclic, etc., depending upon the nutrient and/or light conditions

According to particular aspects, during algal growth within the inventive system, both heterotrophic growth and autotrophic growth may occur. In addition, heterotrophic growth may occur irrespective of the depth of the culture medium within the system or vessel, and/or whether or not the culture is illuminated. According to further embodiments, the inventive symbiotic co-cultivation systems allow for the algae to grow both heterotrophically and autotrophically, and where the relative contribution of each are dependent upon the growth conditions (e.g., nutrients, light, temperature, etc).

### Cultivation/Culture Vessels, Systems, and Methods

Certain aspects of the invention are directed to vessel designs and to methods and systems utilizing vessels for sustainable symbiotic co-culture as disclosed herein. In certain embodiments, the culture vessel comprises a photobioreactor apparatus designed to contain a liquid medium. A diverse number of different designs and types may be employed to practice the disclosed symbiotic co-culture methods including, but not limited to fully or semi-automated artificial bioreactors in both open (to the environment) and closed configurations, and manually operated bioreactors in both open and closed configurations.

A “vessel,” as used herein, refers to an apparatus or structure suitable for retention of culture medium. Preferably, the vessel comprises integral means (e.g. ports, valves, etc., as disclosed herein) for introduction of growth medium (e.g., surface water, ground water, etc.), circulation of growth medium (e.g., pumps, gravity, gasification/aeration means, etc.), filtering mechanism (e.g., porous filters, sand, screens, gravity filters etc.), and is adaptable to a light source (natural sunlight and/or artificial). Typically, the vessels are inoculated to comprise at least one species of algae, where the vessels are adaptable to interface with a source of light capable of driving photosynthesis. For example, the vessels may have at least one surface or at least a portion of a surface that is partially transparent to light of a wavelength suitable for driving photosynthesis (e.g., light of a wavelength between about 400-700 nm). In particular aspects, the term “vessel” refers to photobioreactors. In further aspects the vessel is constructed with any material, including but not limited to stainless steel, iron, fiber glass, glass, cement, plastic, rock, and soil. In still further aspects, the vessel is any shape, size, or depth. According to further aspects, the depth of the vessel is about 10 cm to about 2000 cm, with preferred depths from about 30 cm to about 1500 cm, as disclosed herein.

In certain embodiments, the vessel or the system is operated as a single batch and/or a sequential batch and/or continuously. As disclosed herein, the vessels are inoculated to establish a symbiotic co-culture, which provides for a sustained biomass growth, and wherein a significant or substantial proportion of the macronutrients supporting the co-culture growth (e.g., at least 5%) are derived from dead algal and microbial cells continuously produced within the co-culture. Additionally, the medium (e.g., surface water, ground water, etc.) may contain at least some level of organic and/or inorganic nutrients (e.g., CaCO₃).

In certain embodiments, floating objects and/or devices configured to be partially submerged in the liquid medium (e.g. a paddle wheel, screw, pump, aerators, water falls, etc.) may be used to facilitate enhancement of gas-liquid interfacial area and mass transfer. In certain such embodiments, the objects may be transparent such that they also may act to allow penetration of light to greater depths within the media. In some embodiments, elements may be employed to produce surface ripples or even waves that travel laterally or longitudinally within the liquid medium to increase mass transfer between the gas and the liquid.

The cultivation system and/or culturing vessels may be heated and maintained at certain temperatures or temperature ranges suitable or optimal for productivity. These specific, desirable temperature ranges for operation will, of course, depend upon the characteristics of the phototrophic species used within the cultivation systems, the type of culture vessel, etc. Typically, it is desirable to maintain the temperature of the liquid medium between about 5° C. and about 45° C., more typically between about 15° C. and about 37° C., and most typically between about 15° C. and about 25° C. For example, a desirable temperature operating condition for a cultivating system utilizing Chlorella algae could have a liquid medium temperature controlled at about 30° C. during the daytime and about 25° C. during nighttime. In one embodiment, the temperature of the vessel is maintained at about 25° C.

In certain embodiments, the cultivating system utilizes natural sunlight. In alternative embodiments, an artificial light source providing light at a wavelength able to drive photosynthesis may be utilized in supplement to or instead of natural sunlight. For example, a cultivating system utilizing both sunlight and an artificial light source may be configured to utilize sunlight during the daylight hours and artificial light in the night hours, so as to increase the total amount of time during the day in which the cultivation system can convert CO₂ to biomass through photosynthesis.

According to certain embodiments, aeration and/or the addition of other gases (e.g., air, oxygen, carbon dioxide, nitrogen, etc.), can be by bubbling, stirring, diffusing or carrying dissolved gas (e.g., air, oxygen, etc.) in water stream, such as from an aerator, waterfall, or fountain, without algal cell disruption. According to further embodiments, introduction of oxygen or other gases at certain strategic times disperses carbon dioxide into the air (e.g., sparging) which thus regulates and stabilizes the pH. As appreciated in the art, as carbon dioxide dissolves in water, it forms carbonic acid and lowers the pH).

Different types of algae may require different light exposure conditions for optimal growth and proliferation. In certain embodiments, particularly those where sensitive algal species are employed, light modification apparatus or devices may be utilized in the construction of the cultivation system according to the invention. Some algae species either grow much more slowly or die when exposed to ultraviolet light. If the specific algae species being utilized in the cultivation system is sensitive to ultraviolet light, then, for example, certain portions of a cover, or alternatively, the entire cover outer and/or inner surface, could be coated or covered with one or more light filters that can reduce transmission of the undesired radiation.

### Exemplary Algal Species; Methods are Broadly Applicable:

The cultivation system, utilizing at least one algae species, at least one aerobic bacteria, and at least one diazotroph is designed to be applicable to a broad spectrum of species. The system settings, conformations, dimensions, sub-systems and contents may be adjusted to allow many types of photosynthetic microorganisms (e.g., algae) in combination with at least one aerobic bacteria and at least one diazotroph to be grown. An exemplary organism is Chlorella protothecoides, a non-motile green microalgae that can switch between phototrophic (photosynthetic) and heterotrophic (feeding on an external carbon source) modes. This microorganism also has the ability to accumulate large amounts of neutral lipids (TAGs) within its cytoplasm that can be used as a feedstock for biofuels production. However, the skilled artisan will realize that many species of algae or other photosynthetic microorganisms have been discovered and characterized and that, in alternative embodiments, a broad spectrum of known species may be grown in the cultivation system, in combination with at least one aerobic bacteria and at least one diazotroph to support sustained symbiotic co-cultures and bio-product generation therefrom. Non-limiting exemplary algal species include Nannochloropsis sp., Nannochloropsis salina, Nannochloropsis occulata, Tetraselmis suecica, Tetraselmis chuii, Botrycoccus braunii, Chlorella sp., Chlorella ellipsoidea, Chlorella emersonii, Chlorella minutissima, Chlorella protothecoides, Chlorella pyrenoidosa, Chlorella salina, Chlorella sorokiniana, Chlorella vulgaris, Chroomonas salina, Cyclotella cryptica, Cyclotella sp., Dunaliella salina, Dunaliella bardawil, Dunaliella tertiolecta, Euglena gracilis, Gymnodinium nelsoni, Haematococcus pluvialis, Isochrysis galbana, Monoraphidium minutum, Monoraphidium sp., Nannochloris sp., Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Skeletonema, Stichococcus bacillaris, Spirulina platensis, or Thalassiosira sp.

According to certain aspects, growth of certain algae species is undesirable. The diversity of algal species can be limited by altering different salt concentrations and/or changing the pH of the growth medium.

### Algal Growth

In certain embodiments, at least one algae, at least one aerobic bacteria and at least one diazotroph within the cultivation system have similar life cycles (i.e., they are born, they grow, die and decay) which repeats within the system. In further embodiments, the dead cells of both bacteria and algae function as biomass or organic nutrients for growing algae in the cultivation systems. According to still further embodiments, the excess biomass is used as organic nutrients for algal cultivation.

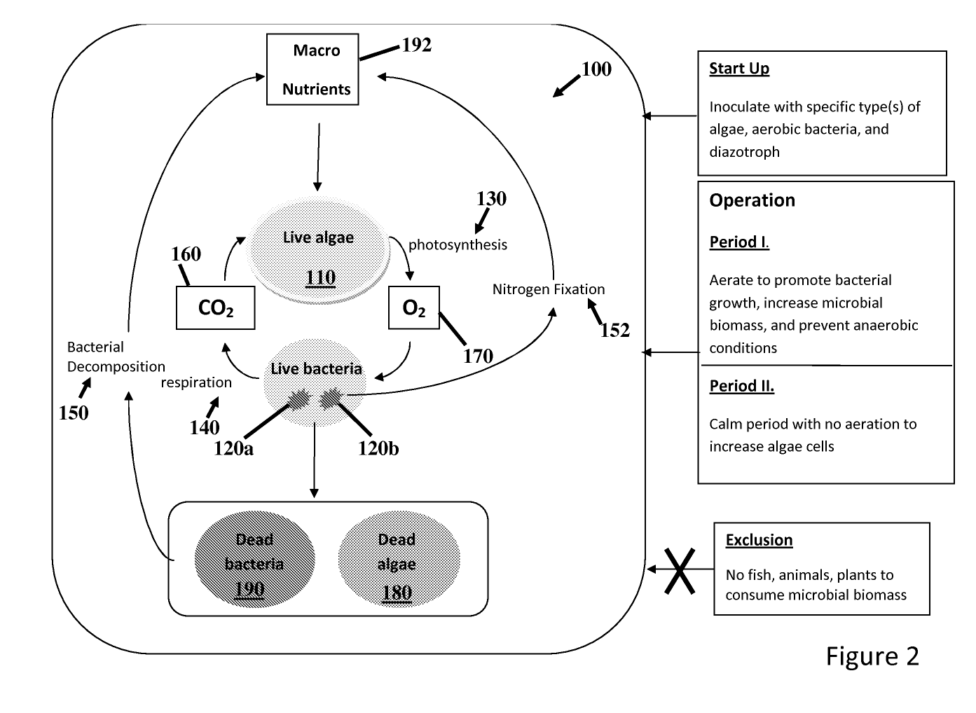

Certain aspects of the invention are directed to the minimal addition of exogenous nutrients to the cultivation system for growing algae because the organic macro nutrients are obtained from sustained symbiosis between algae, aerobic bacteria and diazotrophs (FIG. 2).

In preferred embodiments, at least a certain percentage of macronutrients are derived from dead algae and bacterial debris in the sustained symbiotic cultures, for example, at least 5%, at least 10%, at least 20%, at least 30%, at least 40%, at least 50%, at least 60%, at least 70%, at least 80%, at least 90%, or at least 99% of macronutrients are derived from dead algae and bacterial debris. Preferably, at least 50%, at least 60%, at least 70%, at least 80%, at least 90%, or at least 99% of macronutrients are derived from dead algae and bacterial debris. Most preferably, at least 70% or at least 80% of macronutrients are derived from dead algae and bacterial debris.

According to yet further embodiments, bacteria provide carbon dioxide for algae while the algae produce oxygen as a by-product of photosynthesis, which provides for aerobic respiration of aerobic bacteria. According to yet still further embodiments, diazotrophs supply nitrogen in a bioavailable form (e.g., ammonia, nitrates) that can be readily utilized by both the algae and aerobic bacteria, but at levels, as discuss in detail in Example 7 herein, that surprisingly yet provide for significant enhanced lipid production.

### Algal Harvesting

According to particular aspects, periodically, throughout the co-cultivation of at least one algae with at least one aerobic bacteria and at least one diazotroph, the algae is harvested from the growth medium by skimming the top of the culture and/or collecting from the bottom or from the bulk of the vessel via pumping and filtering. The frequency and extent of algal harvesting is suitable to provide for sustained symbiotic co-culture of the algal, aerobic bacterial and diazotrophic organisms. According to further aspects, only a fraction (e.g., 10%, 15%, 20%, 25%, 30%, 35%, 40%, 45%, or 50%,) of algae in the culture is harvested at any one time (e.g., 10% per day) such that the algae remaining after harvest is sufficient to continuously maintain (e.g., re-establish and/or sustain) the symbiotic co-culture, and enhanced lipid (TAG) production.

### Algal Bio-Products

According to particular aspects, wet or dried algal biomass can be used directly as a solid fuel for use in a combustion device or facility and/or could be converted into a fuel grade oil (e.g., biodiesel) and/or other fuel (e.g., ethanol, methane, hydrogen). The algae also may be used as food supplements for humans and animals. In certain embodiments, at least a portion of the biomass, either dried or before drying, can be utilized for the production of products comprising organic molecules, such as fuel-grade oil (e.g. biodiesel), biocrude, and/or organic polymers. Methods of producing fuel grade oils and gases from algal biomass are well known in the art (e.g., see, Dote, Yutaka, “Recovery of liquid fuel from hydrocarbon rich micro algae by thermo chemical liquefaction,” Fuel. 73:Number 12. (1994); Ben-Zion Ginzburg, “Liquid Fuel (Oil) From Halophilic Algae: A renewable Source of Non-Polluting Energy, Renewable Energy,” Vol. 3, No 2/3. pp. 249-252, (1993); Benemann, John R. and Oswald, William J., “Final report to the DOE: System and Economic Analysis of Micro algae Ponds for Conversion of CO₂ to Biomass.” DOE/PC/93204-T5, March 1996; and Sheehan et al., 1998; each incorporated by reference).

According to particular exemplary aspects, a variety of exemplary beneficial bioproducts can be produced from algae grown in the disclosed symbiotic co-cultivation system. Algae harvested from the cultivation system can be used to produce: e.g., biofuels, biocrude, pharmaceuticals, therapeutics, vitamins, antioxidants, nutraceuticals, cosmetics, cosmeceuticals, bioplastics, food, feed stock, sulfur, and fertilizer.

### Definitions

As used herein, “water” can be from any suitable source, including but not limited to surface water, ground water, brackish water, salt water, sea water, marine water, lake water, river water, wastewater, saline, swamp water, tap water, and sewage.

As used herein, “heterotrophic growth” refers to growth that requires organic compounds for energy and nutrients, such as carbon and nitrogen (e.g., in the absence of photosynthesis). As appreciated in the art, heterotrophic growth of algae results in the majority of energy coming from catabolism of organic compounds rather than photosynthesis.

As used herein, “symbiotically” includes, for example, co-cultivating two or more organisms in an environment wherein each organism benefits from the presence of the other for mutual benefit. Particular symbiotic co-cultures, as discussed herein, are comprised of three organismal components (e.g., algae:aerobic bacteria:diazotroph), or two organismal components (e.g., algae:diazotroph) that exchange nutrients to the mutual benefit of the co-culture, including, for example, oxygen, carbon dioxide and bioavailable nitrogen.

As used herein, “low nutrient addition” refers to the requirement that less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25% of the macronutrient driving growth in the symbiotic culture derives from exogenously added nutrients. Preferably, low nutrient addition refers to the requirement that at least a certain percentage of macronutrients are derived from dead algae and bacterial debris in the sustained symbiotic cultures, for example, at least 5%, at least 10%, at least 20%, at least 30%, at least 40%, at least 50%, at least 60%, at least 70%, at least 80%, at least 90%, or at least 99% of macronutrients are derived from dead algae and bacterial debris. Preferably, at least 50%, at least 60%, at least 70%, at least 80%, at least 90%, or at least 99% of macronutrients are derived from dead algae and bacterial debris. Most preferably, at least 70% or at least 80% of macronutrients are derived from dead algae and bacterial debris. p As used herein, “nutrients” are those inorganic and/or organic chemical compounds that are required and/or beneficial for growth of the algae and/or aerobic bacteria, and/or diazotroph. Nutrients may, for example, consist of or comprise macro and micro nutrients. As used herein, “required nutrients” are those inorganic and/or organic chemical compounds that are required for growth of the algae and/or aerobic bacteria, and/or diazotroph. Required nutrients may, for example, consist of or comprise macro and micro nutrients.

In preferred embodiments, “sustainable growth” means sustained continuous growth, for example, growth that does not vary from that of a sustainable average daily growth rate, by more than about 10%, about 20%, about 30%, about 40%, about 50%, about 60%, about 70%, about 80%, or about 90%. Preferably, sustainable growth means sustained continuous growth that does not vary from that of a sustainable average daily growth rate, by more than about 10%, about 20%, or about 30%. In further preferred aspects the algae growth is substantially continuous growth and/or non-cyclical growth and/or substantially constant low-nutrient conditions, wherein abrupt nutrient level-related changes in growth are avoided.

As used herein “nitrogen source” refers to a source of nitrogen-containing molecules and salts (e.g., ammonia, ammonium, nitrates, nitrogen, etc.) that can be utilized by organisms to produce complex nitrogen-containing structures (e.g., amino acids, DNA, other biological macromolecules, etc.).

As used herein, “nitrogen measurement” includes measuring the amount of nitrogen (e.g., ammonia, ammonium nitrates, nitrogen, etc.) contained within a system, and measuring particular components of total nitrogen. Nitrogen can be measured with respect to any nitrogen-containing molecule, or combination of nitrogen containing molecules, including but not limited to ammonia, ammonium, and nitrates. In certain aspects, measuring nitrogen refers to measuring the total amount of nitrogen (e.g., ammonia, ammonium nitrates, nitrogen, etc.) contained in the culture. Methods for determining nitrogen levels are well known in the art (e.g., total organic nitrogen content, including ammonia and ammonium, can be determined according to the Total Kjeldahl Nitrogen (TKN) method; see, e.g., McKenzie & Wallace, Aust. J. Chem. 7:55, 1954 and Kjeldahl, J., Encyclopedia of Food Science, 439-441, 1983, incorporated herein by reference for these methods).

Adjusting the level of at least one of carbon dioxide, oxygen, and nitrogen (e.g., ammonia, ammonium nitrates, nitrogen, etc.), pH, as used herein, may comprise exogenous addition of carbon dioxide, oxygen, and nitrogen (e.g., ammonia, ammonium nitrates, nitrogen, etc.), or may comprise providing additional cultivation medium containing additional water or adding medium having at least one different level of carbon dioxide, oxygen, or nitrogen (e.g., ammonia, ammonium nitrates, nitrogen, etc.). In certain embodiments, adjusting the level of one of carbon dioxide, oxygen, and nitrogen (e.g., ammonia, ammonium nitrates, nitrogen, etc.) can involve altering the culture by changing the organismal balance between the algae, aerobic bacteria, and dizatropic organism balance. In certain embodiments, adjusting the level of one of carbon dioxide, oxygen, and nitrogen (e.g., ammonia, ammonium nitrates, nitrogen, etc.) can involve adjusting the light source, the pH, the temperature, the ionic strength, the pressure, and medium introduction/flow rate. In certain embodiments, adjusting the level of one of carbon dioxide, oxygen, and nitrogen (e.g., ammonia, ammonium nitrates, nitrogen, etc.) can involve harvesting the algae. In certain embodiments, adjusting the level of one of carbon dioxide, oxygen, and nitrogen (e.g., ammonia, ammonium nitrates, nitrogen, etc.) can involve sparging the culture medium to remove or deplete at least one of carbon dioxide, oxygen, and nitrogen (e.g., ammonia, ammonium nitrates, nitrogen, etc.) from the culture medium.

“Nitrogen-stress,” as used herein, refers to growth conditions and/or cultivation media in which the amount of nitrogen is low or lacking and/or the source of nitrogen is in a form that is not usable (e.g., bioavailable) or less usable by the algal cell component of the symbiotic co-cultures (see, e.g., Flynn, K. J., Marine Ecology Progress Report, 61:297-307, 1990; incorporated herein by reference for its teachings with respect to nitrogen stress responses). For example, the preferred form of nitrogen for algal growth is ammonium or ammonia, and the less-preferred forms of nitrogen are nitrates, nitrites, or elemental nitrogen. In certain embodiments, no nitrogen addition, or supplying a non-preferred or less-preferred form of nitrogen in the cultivation medium induces at least one nitrogen-stress response in the algal component of the symbiotic co-culture. Variation of the relative contributions of various forms of nitrogen in the cultivation medium can be used to affect nitrogen stress responses. In further embodiments, limiting or excluding nitrogen from the growth conditions induces at least one nitrogen-stress response in the algal component of the symbiotic co-culture. The at least one nitrogen stress response includes, but is not limited to the following: low or reduced glutamine to glutamate ratio, low or reduced amino acid to protein ratio, enhanced lipid content, enhanced triacylglycerol (TAG) content, enhanced proportion of lipid in the form of TAGs, accumulation of saturated and monounsaturated fatty acids in triacylglycerols (TAGs) relative to polyunsaturated fatty acids (PUFAs) in TAGs, and depletion of polar lipids. In certain aspects, nitrogen stress can be combined with at least one of carbon and energy (e.g., amount of light) stress.

According to certain exemplary embodiments, the cultivation medium may include sodium nitrate as a nitrogen source in a concentration of about 0.5 mM (starved) to about 5 mM (deprived), or less than 10 mM (considered as a saturating amount for Phaeodactylum tricornutum, for example; see Alonso et al., Phytochemistry 54:461-471, 2000; incorporated herein by reference for its teachings with respect to nitrogen stress responses). In particular embodiments, the concentration of usable nitrogen is that amount of bioavailable nitrogen equivalent to about 2 mM to about 5 mM sodium nitrate, although said bioavailable nitrogen form could be other than sodium nitrate. One of ordinary skill in the art will be able to readily determine nitrogen stress conditions for the particular algal component used in the disclosed symbiotic co-cultures, without undue experimentation. Typically, in order to sustain at least one nitrogen-stress response in the algal component of the symbiotic co-culture, an amount of nitrogen equivalent to less than about 10 mM sodium or potassium nitrate is used. Preferably, an amount of nitrogen equivalent to less than about 5 mM sodium or potassium nitrate is used. More preferably, an amount of nitrogen equivalent to less than about 2 mM sodium or potassium nitrate is used. For example, very low nutrient media, as used herein, comprises 100 g of KNO₃ (i.e., 1 mM), 10 g of KH₂PO₄, 10 g of Na₂HPO₄, 1000 g of NaHCO₃, 1.5 g Fe-EDTA, 0.36 g of MnCl₂*4H₂O, 0.4 g of MgSO₄*7H₂O, 0.5 g of H₃BO₃, 0.3 g of ZnSO₄*7H₂O, 0.1 g of Na₂MoO₄*2H₂O, 0.016 g of CuSO₄*5H₂O, and 0.01 g Co(NO₃)₂*6H₂O per 1000 L of surface water. By contrast, high nutrient growth media, as used herein, comprises 1.250 g of KNO₃, (i.e., 12.3 mM), 1.350 g of NaNO₃, 1.250 g of KH₂PO₄, 0.500 g of K₂HPO₄, 0.010 g Na₂HPO₄, 1.000 g of NaHCO₃, 0.360 g of MgSO₄*7H₂O, 0.384 g of CaCl₂, and 1.680 g of NaHCO₃ per 1 L of water. Therefore, if required to adjust or augment the nitrogen content of the symbiotic co-culture medium, a suitable amount of either low, or high nutrient growth media, as used herein, may be added to the co-culture medium to provide nitrogen, while maintaining nitrogen stress conditions. In preferred aspects, the at least one diazotroph, provides a sustainable level of bioavailable nitrogen in the symbiotic co-culture, and significantly in preferred aspects, the cultivation medium is suitable to induce at least one nitrogen stress response in the algal cells cultured therein, and the diazotroph component is maintained in an amount sufficient to sustainably attenuate at least one nitrogen stress response in the symbiotically co-cultivated algal cells.

The term “photosynthetic organism”, “phototrophic organism”, or “biomass” as used herein, includes all organisms capable of photosynthetic growth, such as plant cells and micro-organisms (including algae, euglena and lemna) in unicellular or multi-cellular form that are capable of growth in a liquid phase.

According to preferred aspects, initially establishing the algae, aerobic bacteria, diazotroph co-culture comprises use of a suitable seed culture or inoculum to provide for an initial symbiotic biomass ratio of the algae, aerobic bacteria, diazotroph co-culture components of the symbiotic growth system. The initial inoculum is selected to provide for a subsequent establishment of a sustained symbiotic biomass ratio of the algae, aerobic bacteria, diazotroph co-culture components. Exemplary and preferred initial symbiotic biomass ratios and sustained symbiotic biomass ratios are provided in Table 5, along with exemplary preferred ranges for each organismal component of the ratios.

For example, an initial inoculum biomass ratio of algae:aerobic bacteria:diazotroph of 100:1.6:0.18 (wt. %) (see top row of Table 5), respectively, typically provides sufficient numbers of organisms of each type to subsequently establish a symbiotic co-culture according to the present invention, which includes a significant sustained fraction of harvestable algae, as well as a significant sustained fraction of dead organisms that typically form clumps or sludge with live organisms of the co-culture, and wherein the dead organisms substantially provide sustained macronutrients (and micro to some extent) for the established sustained symbiotic co-culture for algal production (e.g., of biofuels, etc.).

According to further aspects, the initial relative ratios of organisms (e.g., the inoculum ratio of 100:1.6:0.18 (wt. %)) may be similar or may vary somewhat from the subsequent sustained symbiotic biomass ratio of the ‘established’ symbiotic co-culture. Exemplary, preferred ranges for the initial and sustained biomass ratios as also given in Table 5 (bottom two rows). Depending on the particular growth conditions and organisms used, one of the three organism types may differentially contribute more prominently to the established live biomass ratio, or differentially contribute more prominently to the established dead biomass ratio.

TABLE 5
Exemplary preferred symbiotic ratios and symbiotic ratio ranges.

| Initial Inoculum ratio; | Sustained symbiotic ratio | Sustained symbiotic ratio |
|---|---|---|
| (Algae:aerobic | (not including dead | (including dead |
| bacteria:diazotroph) (wt. %) | biomass) (wt. %) | biomass) (wt. %) |
| 100:1.6:0.18 | 100:1.6:0.18 | 110:10:1.5 |
| 10:1.6:18 | 100:25:18 | 150:50:15 |

Exemplary preferred ranges

| | | |
|---|---|---|
| 50-500:0.8-80:0.09-9 | 50-500:0.8-80:0.09-9 | 55-550:5-50:0.75-7.5 |
| 10-1000:0.16-160:0.018-18 | 10-1000:0.16-160:0.018-18 | 15-1100:1-100:0.15-15 |

The exemplary initial inoculum biomass ratio of algae:aerobic bacteria:diazotroph of 100:1.6:0.18 (wt. %) (see top row of Table 5), is illustrative of the fact, according to aspects of the present invention, that there is less need for large amounts of aerobic bacteria in the initial cultures, and the fact that bacterial growth is relatively rapid compared to algal growth, such that the initial bacteria level is strategically selected to ‘grow into equilibrium’ with the algal growth of the co-culture. The higher relative proportions of bacteria in the sustained symbiotic ratio including the dead biomass, is illustrative of the fact that bacteria, according to particular aspects, tend to differentially contribute to the dead biomass of the inventive sustained symbiotic cultures.

For inventive two component continuous symbiotic co-cultures, preferred exemplary initial inoculum biomass ratios and sustained symbiotic biomass ratios of algae:diazotroph are provided by the respective three component ratios in Table 5 (i.e., by deleting the middle term of the three component ratio). The same applies with respect to choice of two component organisms (i.e., algae:diazotroph) from those organism listed in Tables 1-4.

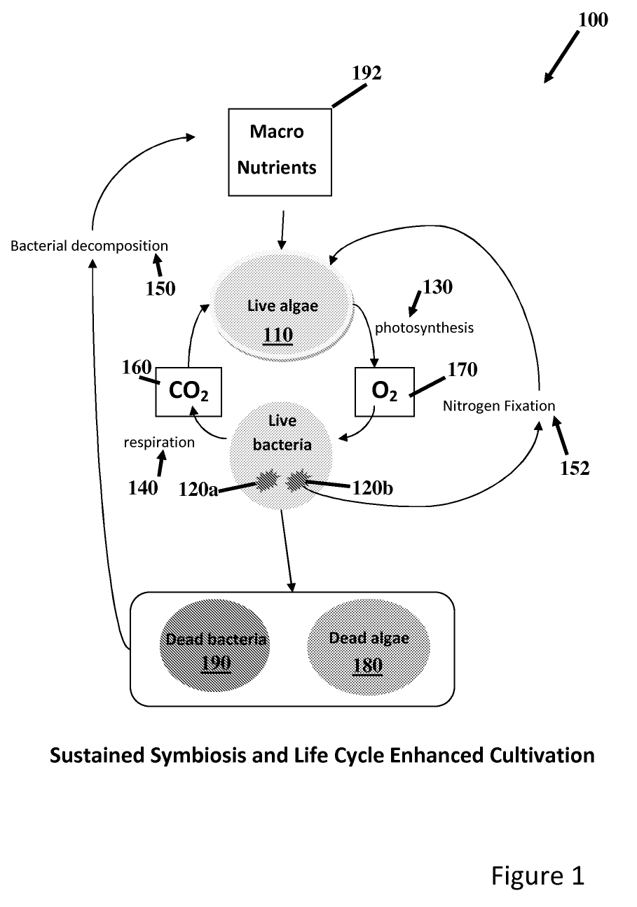

According to particular exemplary aspects, FIG. 1 illustrates the conceptual basis underlying Applicant's sustained co-culture and lifecycle-enhanced cultivation of algae. In an open system 100, at least one inoculated algae 110, at least one aerobic bacteria 120a, and at least one diazotroph 120b exist in a symbiotic relationship. In this system 100, several processes are occurring: photosynthesis 130, respiration 140, bacterial and/or algal decomposition 150, nitrogen fixation 152 stress-attenuation mediated by a diazotroph (as disclosed herein), and heterotrophic and/or autotrophic algal growth. For photosynthesis 130, algae 110 utilize carbon dioxide 160, which is a by-product of aerobic bacteria 120a respiration 140, to produce oxygen 170. In respiration 140, the aerobic bacteria 120a utilize oxygen 170 that has been produced from algal photosynthesis 130 to produce carbon dioxide 160. Eventually, for example, when a fraction of the aerobic bacteria 120a and algae 110 die and become dead algae 180 and dead bacteria 190, the aerobic bacteria 120a (and/or the diazotroph 120b and/or algae 110) decompose 150 the dead algae 180 and dead bacteria 190, to produce organic macro-nutrients 192, which are then used by the living algae 110 and/or aerobic bacteria 120a (and/or diazotroph 120b) to grow. The at least one diazotroph 120b, provides a sustainable level of bioavailable nitrogen in the symbiotic co-culture, and significantly in preferred aspects, the cultivation medium is suitable to induce at least one nitrogen stress response in the algal 110 cells cultured therein, and the diazotroph 120b component is maintained in an amount sufficient to sustainably attenuate at least one nitrogen stress response in the symbiotically co-cultivated algal 110 cells.

According to particular exemplary aspects, FIG. 2 summarizes the process of sustained co-culture and life cycle-enhanced cultivation of algae. The process can be divided into two parts: Start up and Operation. The Start Up portion involves inoculating the open system 100 with at least one specific type of algae 110, at least one specific type of aerobic bacteria 120a, and at least one specific type of diazotroph 120b. The Operation portion is divided into two periods: Period I and Period II. Period I relates to a process wherein, the system 100 is aerated periodically to promote bacterial growth, to increase microbial biomass, and to prevent anaerobic conditions. Period II relates to a period of no aeration. In preferred embodiments, during this period of no aeration, the algae 110 cells accumulate. Preferably, including in open systems other plants, animals, insects, fish, etc., are excluded from the system 100 to preclude unwanted consumption of the microbial and algal biomass being produced within the inventive symbiotic co-cultures.

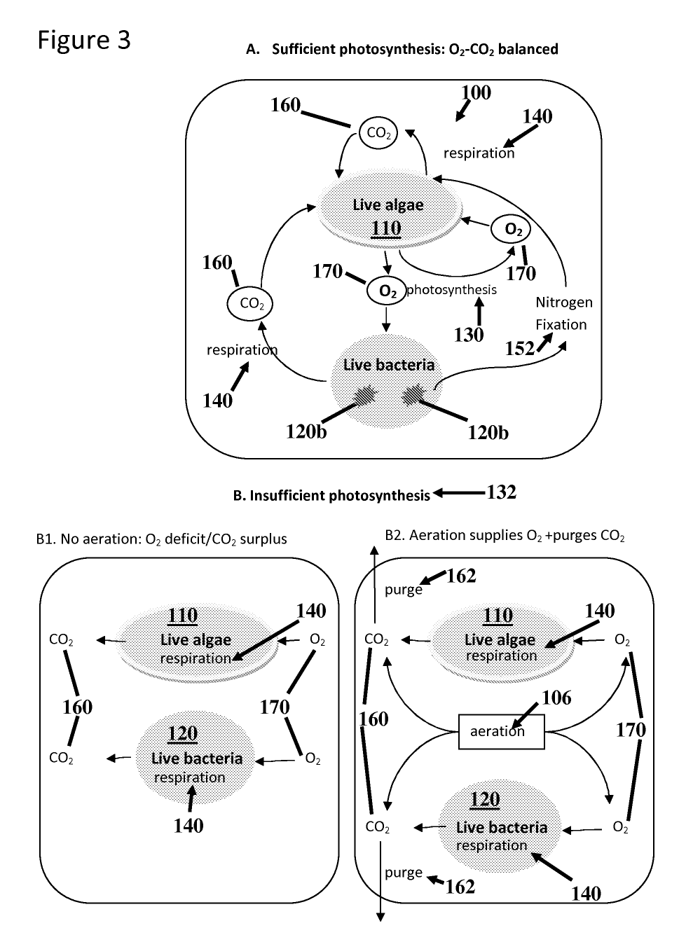

According to particular exemplary aspects, FIG. 3 demonstrates two exemplary scenarios occurring in the system 100, as disclosed in FIG. 1. Scenario A applies when there is sufficient photosynthesis 130 to keep an appropriate balance of oxygen 170 and carbon dioxide 160 for algae growth. Scenario B applies when there is no photosynthesis 132 or insufficient photosynthesis 132 (B1). In scenario B1, both the algae 110 and aerobic bacteria 120 undergo respiration 140 (e.g., use oxygen 170 to produce carbon dioxide 160_due to the lack of sufficient photosynthesis. In addition, with insufficient photosynthesis 132 (B1), the percentage of algae growing heterotropically increases. In scenario B2, aeration 106 is introduced to ensure that there is sufficient oxygen 170 for algae growth and/or to purge 162 carbon dioxide 160. In both of the exemplary scenarios, as shown in FIGS. 3A and 3B, nitrogen-fixing occurs normally, (e.g., the diazotrophs continue to fix nitrogen and the algae 110 and aerobic bacteria 120 continue to utilize the fixed nitrogen as a nitrogen source). In preferred aspects, the cultivation medium is suitable to induce at least one nitrogen stress response in the algal 110 cells cultured therein, and the diazotroph 120b component is maintained in an amount sufficient to sustainably attenuate at least one nitrogen stress response in the symbiotically co-cultivated algal 110 cells.

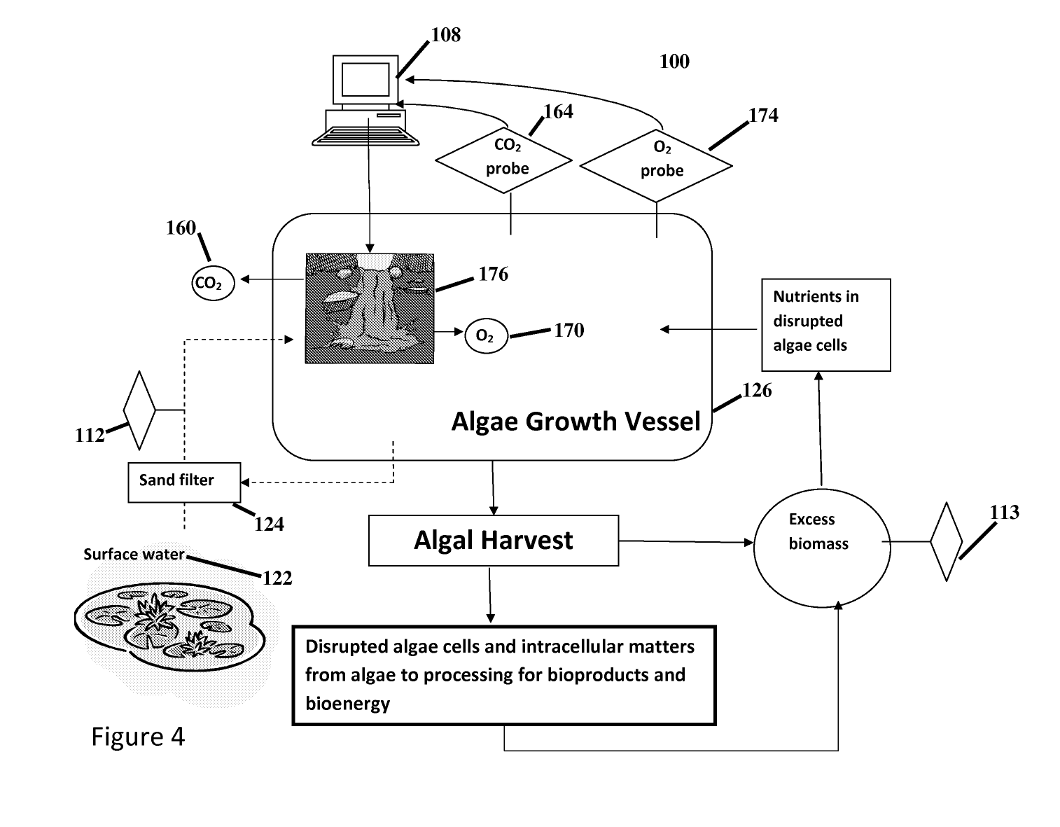

According to particular exemplary aspects, FIG. 4 shows an automated cultivation system 100 for growing algae that produces bioproducts and/or bioenergy for monitoring the system disclosed in FIG. 1. The automated cultivation system 100 is controlled via a computer 108 that receives signals from both an oxygen probe 174 and a carbon dioxide probe 164. According to further exemplary aspects, water 122 from any suitable source (e.g., surface water, ground water, etc.) passes through a sand filter 124 before filling the cultivating vessel 126 (e.g., algae growth vessel). An analyzer 112 checks for the composition and amount of nutrients in the water 122. The water 122, in the cultivating vessel 126 is inoculated with at least one algae, at least one aerobic bacteria, and at least one diazotroph at a suitable predetermined initial biomass ratio, and the inoculum is allowed to grow to achieve a symbiotic co-culture relationship. Throughout the co-culturing, the oxygen probe 174 and a carbon dioxide probe 164 continuously or periodically monitor the oxygen 170 and/or carbon dioxide 160 level(s). If the oxygen 170 level falls below an acceptable value or if the carbon dioxide 160 level exceeds the limitation, then the oxygen probe 174 and/or a carbon dioxide probe 164 send signals to the computer 108 which triggers the activation of an aeration device or means 176 (e.g., a waterfall, bubbler, stirrer, diffuser, fountain, etc.). Once the oxygen 170 and carbon dioxide 160 levels are back to the desired levels, the computer 108 deactivates the aeration device or means 176. As the algae, aerobic bacteria, and diazotroph grow in the vessel 126, a proportion of the biomass comprising dead algae and bacteria cells begin to accumulate. These dead cells then are decomposed by the bacteria and provide nutrients to the remaining living organisms of the co-culture, including the algae. According to further exemplary aspects, periodically, a sustainable percentage of the algae is harvested from the vessel and processed to obtain a biomass product. According to still further exemplary aspects, if the nutrients from dead cells in the vessel are not sufficient to sustain growth, then some portion of post extraction algae can be recycled back into the algae growth vessel 126 to provide additional nutrients to support proper growth. An analyzer 113 checks for the composition and amount of nutrients in the algae. In preferred aspects, the cultivation medium is suitable to induce at least one nitrogen stress response in the algal 110 cells cultured therein, and the diazotroph 120b component is maintained in an amount sufficient to sustainably attenuate at least one nitrogen stress response in the symbiotically co-cultivated algal 110 cells. In additional aspects, therefore, nitrogen levels are monitored and adjusted if required to provide for sustainable attenuatation of the at least one nitrogen stress response in the symbiotically co-cultivated algal 110 cells.

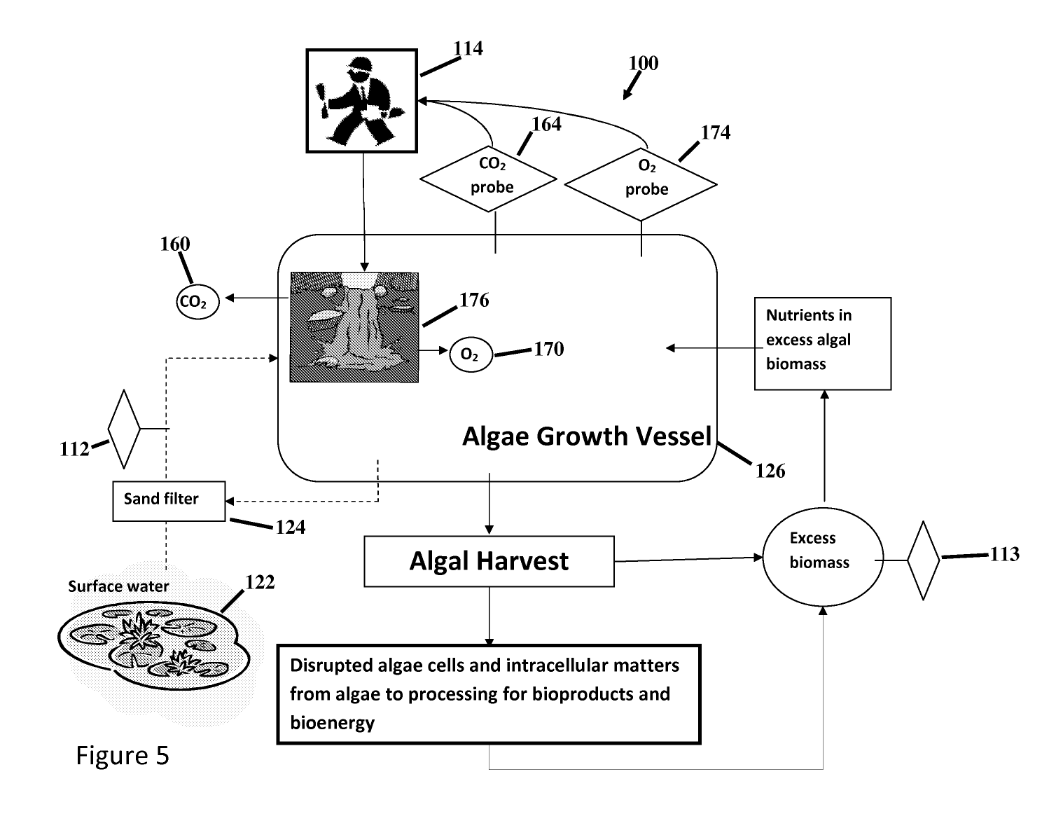

According to particular exemplary aspects, FIG. 5 shows another embodiment of the disclosed invention described in FIG. 4, wherein the computer is omitted and instead a technician 114 manually controls the symbiotic co-cultivation system 100 for growing algae that produces bioproducts and/or bioenergy.

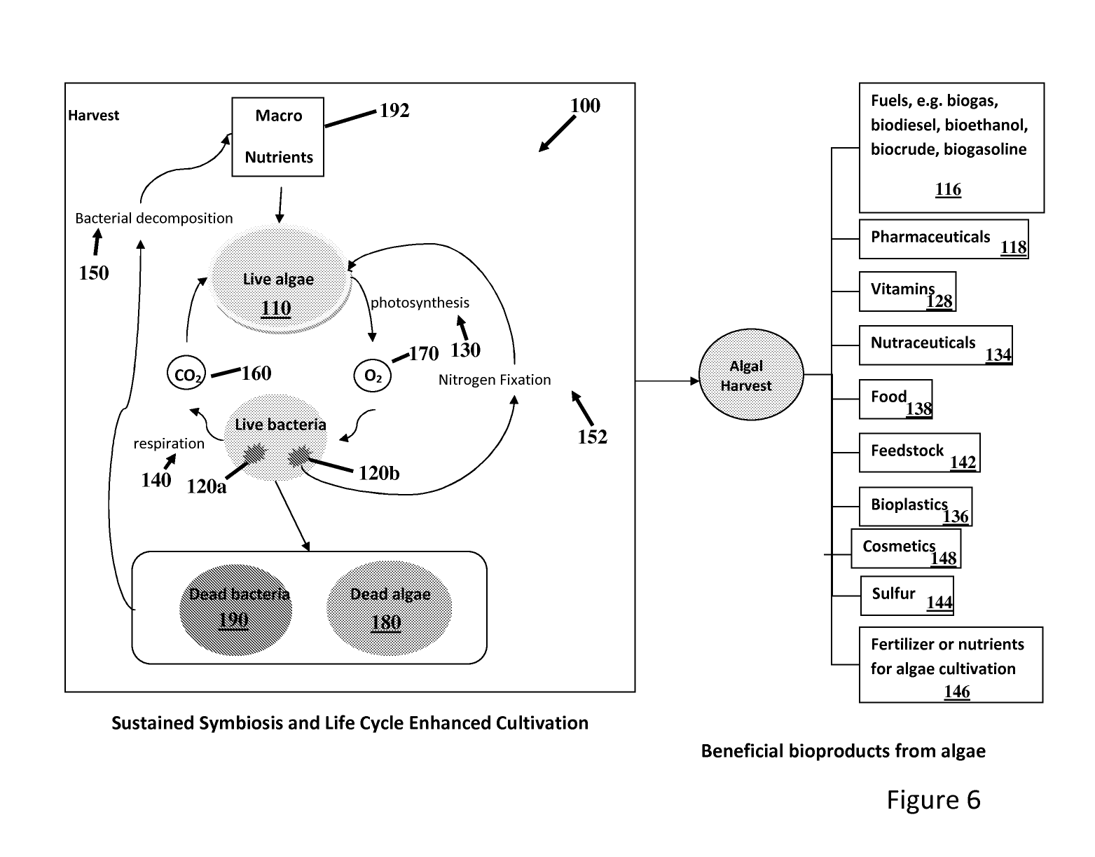

According to particular exemplary aspects, FIG. 6 displays, a variety of exemplary beneficial bioproducts that can be produced from algae grown in the disclosed symbiotic co-cultivation system. In the open algal growth system 100 disclosed herein, the algae 110 harvested from the system 100 can be used to produce: biofuels 116, pharmaceuticals 118, therapeutics, antioxidants, vitamins 128, nutraceuticals 134, bioplastics 136, food 138, feed stock 142, cosmetics 148, cosmeceuticals, sulfur 144, and fertilizer 146.

### Optional use of at Least One Genetically Engineered Algae, Aerobic Bacteria, or Diazotroph:

In certain embodiments, the algae used (e.g., to produce bio-product or biofuels) in the symbiotic co-cultures may be genetically engineered (e.g., mutant, transgenic, etc.) to contain one or more nucleic acid sequences that enhance production, directly or indirectly, of a particular bio-product, or provide other desired characteristics beneficial for improved algal symbiotic co-culture, growth, yield, product quality, harvesting efficiency, processing efficiency or utilization efficiency. Methods of mutagenesis and/or of stably transforming algal species and compositions comprising isolated, modified, mutant, altered, etc., nucleic acids for use in the present invention are well known in the art, and it will be generally appreciated that any such methods and compositions may be readily used, given the teachings disclosed herein, in the practice of the present invention without the need for undue experimentation. Exemplary transformation methods of use may include microprojectile bombardment, electroporation, protoplast fusion, PEG-mediated transformation of protoplasts, DNA-coated silicon carbide whiskers or use of viral mediated transformation, or vortexing protoplasts with glass beads in a solution containing the DNA to be transformed into the algal cell (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017, incorporated herein by reference and particularly those portions relating to these and other suitable transformation method teachings).

For example, U.S. Pat. No. 5,661,017 discloses methods for algal transformation of chlorophyll C-containing algae, such as the Bacillariophyceae, Chrysophyceae, Phaeophyceae, Xanthophyceae, Raphidophyceae, Prymnesiophyceae, Cryptophyceae, Cyclotella, Navicula, Cylindrotheca, Phaeodactylum, Amphora, Chaetoceros, Nitzschia or Thalassiosira. Compositions comprising useful nucleic acids, such as acetyl-CoA carboxylase, are also disclosed in U.S. Pat. No. 5,661,017, along with suitable expression vectors.

In various embodiments, a selectable marker may be incorporated into a nucleic acid or vector to facilitate selection of transformed algae, or for maintenance of transformed algae. Suitable selectable markers may include, but are not limited to at least one selected from, neomycin phosphotransferase, aminoglycoside phosphotransferase, aminoglycoside acetyltransferase, chloramphenicol acetyl transferase, hygromycin B phosphotransferase, bleomycin binding protein, phosphinothricin acetyltransferase, bromoxynil nitrilase, glyphosate-resistant 5-enolpyruvylshikimate-3-phosphate synthase, cryptopleurine-resistant ribosomal protein S14, emetine-resistant ribosomal protein S14, sulfonylurea-resistant acetolactate synthase, imidazolinone-resistant acetolactate synthase, streptomycin-resistant 16S ribosomal RNA, spectinomycin-resistant 16S ribosomal RNA, erythromycin-resistant 23S ribosomal RNA and methyl benzimidazole-resistant tubulin.

In additional embodiments, the aerobic bacteria component of the symbiotic co-culture may be a mutant and/or genetically engineered (e.g., transgenic) organism, containing one or more modified, mutant, altered, etc., nucleic acid sequences that enhance production, directly or indirectly, of a particular bio-product, or provide other desired characteristics beneficial for improved algal symbiotic co-culture, growth, yield, product quality, harvesting efficiency, processing efficiency or utilization efficiency. Suitable methods of stably transforming bacterial species and compositions comprising suitable isolated nucleic acids and expression vectors are well known in the art and it will be generally appreciated that any suitable methods and compositions may be used in the practice of the present invention without a requirement for undue experimentation. Exemplary suitable transformation methods may include, but are not limited to, at least one of microprojectile bombardment, electroporation, PEG-mediated transformation of bacteria, DNA-coated silicon carbide whiskers or use of viral mediated transformation, and vortexing bacteria with glass beads in a solution containing the DNA to be transformed into the bacterial cell.

In further embodiments, the diazotrophic organism component of the symbiotic co-culture may be a mutant and/or genetically engineered (e.g., transgenic) organism, containing one or more modified, mutant, altered, etc., nucleic acid sequences that enhance production, directly or indirectly, of a particular bio-product, or provide other desired characteristics beneficial for improved algal symbiotic co-culture, growth, yield, product quality (e.g., lipid composition and/or structure, etc.), harvesting efficiency, processing efficiency or utilization efficiency. Suitable methods of stably transforming diazotrophic species and compositions comprising suitable isolated nucleic acids and expression vectors are well known in the art and it will be generally appreciated that any suitable methods and compositions may be used in the practice of the present invention without a requirement for undue experimentation. Exemplary suitable transformation methods may include, but are not limited to, at least one of microprojectile bombardment, electroporation, protoplast fusion, PEG-mediated transformation of protoplasts, DNA-coated silicon carbide whiskers, use of viral mediated transformation, and vortexing protoplasts with glass beads in a solution containing the DNA to be transformed into the algal cell (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017, incorporated herein by reference and particularly those portions relating to these and other suitable transformation methods).

In certain embodiments, at least one of the co-culture organism components comprises at least one modified, mutant, altered, transformed, transfected, recombinant, etc., nucleic acid sequence that enhances production, directly or indirectly, of a particular bio-product, or that provides other desired characteristics beneficial for improved algal symbiotic co-culture, growth, yield, product quality (e.g., lipid composition and/or structure, etc.), harvesting efficiency, processing efficiency or utilization efficiency, etc.

In further embodiments, the diazotrophic organism component of the symbiotic co-culture may be a mutant and/or genetically engineered (e.g., transgenic) organism, containing one or more modified, mutant, altered, etc., nucleic acid sequences that enhance production, directly or indirectly, of a particular bio-product, or provide other desired characteristics beneficial for improved algal symbiotic co-culture, growth, yield, product quality (e.g., lipid composition and/or structure, etc.), harvesting efficiency, processing efficiency or utilization efficiency. Suitable methods of stably transforming diazotrophic species and compositions comprising suitable isolated nucleic acids and expression vectors are well known in the art and it will be generally appreciated that any suitable methods and compositions may be used in the practice of the present invention without a requirement for undue experimentation. Exemplary suitable transformation methods may include, but are not limited to, at least one of microprojectile bombardment, electroporation, protoplast fusion, PEG-mediated transformation of protoplasts, DNA-coated silicon carbide whiskers, use of viral mediated transformation, and vortexing protoplasts with glass beads in a solution containing the DNA to be transformed into the algal cell (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017, incorporated herein by reference and particularly those portions relating to these and other suitable transformation methods).

In certain embodiments, at least one, at least two, or all three of the three co-culture organism components (algae:aerobic bacteria:diazotroph) comprises at least one modified, mutant, altered, etc., nucleic acid sequence that enhances production, directly or indirectly, of a particular bio-product, or provides other desired characteristics beneficial for improved algal symbiotic co-culture, growth, yield, product quality (e.g., lipid composition and/or structure, etc.), harvesting efficiency, processing efficiency or utilization efficiency, etc.

### Example 1

### Material and Methods

Co-culture growth conditions. Surface water was inoculated with at least one algal species, at least one aerobic bacteria, and at least one diazotrophic species in a range ratio of 10-1000:0.16-160:0.018-18, respectively (for specific ratios see examples disclosed herein). If needed (e.g., the algae is growing poorly) and/or to increase algae growth, a very low nutrient medium (as defined herein) was added to the surface water. The depth of the growth medium was kept constant at 40 cm by manually measuring the depth of the growth medium and adding growth medium sufficient to establish the proper depth, or the depth was adjusted automatically with a float ball. The temperature was maintained between 25-30° C. by adding cold water to the medium if the temperature is higher than 30° C. or heating by exchanging heat with waste steam if the temperature is lower than 25° C. Typically, the level of CO₂ was maintained within a range of about 1200 mg/L to about 1400 mg/L. Typically, the level of O₂ was maintained within a range of about 6 mg/L to about 50 mg/L. Typically, the level of nitrogen (see definition of nitrogen herein under “Definitions”) was maintained within a range of about 14 mg/L to about 18 mg/L. Typically, the pH is kept at a value between about pH 6.5 and pH 7.8 for optimal growth.

Nitrogen measurement. Total organic nitrogen content, including ammonia and ammonium, was determined according to the Total Kjeldahl Nitrogen (TKN) method. For specific technique, see McKenzie, H. A. & H. S. Wallace. 1954. “The Kjeldahl determination of nitrogen: A critical study of digestion conditions.” Aust. J. Chem. 7:55 and “Kjeldahl, J. (1883). Determination of protein nitrogen in food products.” Encyclopedia of Food Science, 439-441.

Adjusting CO₂, O₂, and nitrogen levels. For optimal growth, the levels of CO₂, O₂, and nitrogen in the growth medium, were monitored either automatically via computer or manually and adjusted, if necessary, to maintain growth. To adjust the level of one of carbon dioxide, oxygen, and nitrogen, one of several methods was utilized. First, exogenous carbon dioxide, oxygen, and nitrogen can be added to the co-culture. Exogenous addition of oxygen was accomplished by aerating the co-culture, which involves the addition of oxygen via waterfall and/or bubbler and/or stirrer and/or fountain. Second, additional cultivation medium containing additional water or having at least one different level of carbon dioxide, oxygen, or nitrogen is used. Third, altering the organismal balance between the algae, aerobic bacteria, and diazotrophic results in adjusting the carbon dioxide and/or oxygen and/or nitrogen levels. Fourth, adjusting the light source and/or the pH and/or the temperature and/or the ionic strength and/or the pressure and/or flow rate of the surface water results in adjusting the level of one of carbon dioxide, oxygen, and nitrogen. Fifth, harvesting the algae results in adjusting the level of one of carbon dioxide, oxygen, and nitrogen. Last, sparging one of carbon dioxide, oxygen, and nitrogen from the culture medium results in adjusting the level of one of carbon dioxide, oxygen, and nitrogen.

Adjusting pH levels. In addition, the pH was monitored either automatically via computer or manually and adjusted if necessary to maintain the pH at a value between about pH 6.5 and pH 7.8. To adjust the pH level one of several methods was utilized. First, exogenous carbon dioxide, oxygen, and nitrogen is added to the co-culture. Exogenous addition of oxygen was accomplished by aerating the co-culture, which involves the addition of oxygen via waterfall and/or bubbler and/or stirrer and/or fountain. Second, additional cultivation medium containing additional water having a lower or higher pH is added. Third, altering the organismal balance between the algae, aerobic bacteria, and dizatropic results in adjusting the pH. Fourth, adjusting the light source and/or the temperature and/or the ionic strength and/or the pressure and/or flow rate of the surface water results in adjusting the pH level. Fifth, harvesting the algae results in adjusting the pH level. Last, sparging one of carbon dioxide, oxygen, and nitrogen from the culture medium results in adjusting the pH level.

Very low nutrient media. Very low nutrient media was 100 g of KNO₃, 10 g of KH₂PO₄, 10 g of Na₂HPO₄, 1000 g of NaHCO₃, 1.5 g Fe-EDTA, 0.36 g of MnCl₂*4H₂O, 0.4 g of MgSO₄*7H₂O, 0.5 g of H₃BO₃, 0.3 g of ZnSO₄*7H₂O, 0.1 g of Na₂MoO₄2H₂O, 0.016 g of CuSO₄*5H₂O, and 0.01 g Co(NO₃)₂*6H₂O per 1000 L of surface water.

High nutrient growth media. High nutrient growth media was 1.250 g of KNO₃, 1.350 g of NaNO₃, 1.250 g of KH₂PO₄, 0.500 g of K₂HPO₄, 0.010 g Na₂HPO₄, 1.000 g of NaHCO₃, 0.360 g of MgSO₄*7H₂O, 0.384 g of CaCl₂, and 1.680 g of NaHCO₃ per 1 L of water. The pH is adjusted to 6.8.

Algae harvesting. Algae were harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10 percent) of algae in the culture was harvested such that the algae remaining after harvest was sufficient to re-establish and/or sustain the symbiotic co-culture.

### Example 2

### Continuous, Symbiotic Co-Cultures of Chlorella vulgaris, Chlorella sp. D101, Bacillus sp D320, Rhodobacter sphaeroides, Rhodobacter sp. D788, and Spirulina maxima, Spirulina sp. D11 were Established and Maintained for Continuous Sustained Symbiotic Growth

Example Overview. In this working Example 2, a continuous, symbiotic sustainable co-culture was established in a suitable culture vessel using an exemplary algal species, two exemplary aerobic bacterial species, and an exemplary diazotroph (e.g., diazotrophic bacteria).

Specifically, a production culture for continuous and symbiotic algal growth was established by inoculating surface water with algal species Chlorella vulgaris, Chlorella sp. D101, two aerobic bacterial species Rhodobacter sphaeroides, Rhodobacter sp. D788 and Bacillus sp D320, and diazotrophic bacterial species Spirulina maxima, Spirulina sp. D11. The production culture vessel was a rectangular open plastic container having the dimensions of 1.25×2.75 m². Growth medium (surface water) was added via batch flow to the vessel to a depth of 40 cm and circulated by using a pump. Throughout the experiment, the depth of the growth medium was kept constant at 40 cm by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1.

The levels of CO₂, O₂, and nitrogen in the growth medium, were monitored manually and adjusted, if necessary to maintain growth. Typically, the level of CO₂ was maintained within a range of about 1200 mg/L to about 1400 mg/L. Typically, the level of O₂ was maintained within a range of about 6 mg/L to about 50 mg/L. Typically, the level of nitrogen (see definition of nitrogen herein under “Definitions”) was maintained within a range of about 14 mg/L to about 18 mg/L. The different methods used for adjusting and/or maintaining the CO₂, O₂, and nitrogen levels are discussed herein under “Example 1.” In addition, the pH was monitored manually and adjusted if necessary to maintain the pH at a value between about pH 6.5 and pH 7.8 to optimize growth. Methods for measuring and adjusting the pH are discussed herein under “Example 1.” The pH was measured by a pH probe and adjusted by addition of calcium carbonate and CO₂. In addition, throughout the growth cycle of the co-culture, the relative amounts of the algal, aerobic bacterial and diazotrophic organisms were monitored, via plate count and direct count using a microscope, and were adjusted, when necessary, by supplementing the growth medium with either CO₂, O₂, nitrogen, or air as appropriate to provide for sustained symbiotic growth with minimal addition of exogenous nutrients. Additionally, when required, particular organism of the co-culture were supplemented by addition of the respective organism(s) to provide for maintaining the sustained co-culture. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively. Representative, suitable ranges for the ratios or proportions of the algal, aerobic bacterial and diazotrophic organisms are provided under Example 1.

Periodically, throughout the growth cycle Chlorella vulgaris, Chlorella sp. D101 was harvested from the growth medium by skimming the top of the culture and/or collecting from the bottom and/or from the bulk of the vessel via pumping and/or filtering. The frequency and extent of algal harvesting was suitable to provide for sustained symbiotic co-culture of the algal, aerobic bacterial and diazotrophic organisms. Typically, only a fraction (e.g., 10%, 25%, 30%, 35%, 40%, 45%, or 50%) of algae in the culture was harvested such that the algae remaining after harvest is sufficient to re-establish and/or sustain the symbiotic co-culture. The continuous, symbiotic co-culture of this Example was maintained for at least 1 year, before ending the growth protocol.

### Results

In this Example, a high yield of dry weight algae (e.g., 0.45 g/L/day) was sustainable, by establishing a symbiotic co-culture of an aerobic bacteria and a diazotroph with the algae as described herein. See Example 6.

### Example 3

### Continuous, Symbiotic Co-Cultures of Chlorella vulgaris, Chlorella sp. D101, Bacillus sp D320, Rhodobacter sphaeroides, Rhodobacter sp. D788, Methanobacteria sp D422, and Spirulina maxima, Spirulina sp. D11 were Established and Maintained for Continuous Sustainable Symbiotic Growth

Example Overview. In this working Example 3, a continuous, symbiotic sustainable co-culture, similar to Example 2, was established in a suitable culture vessel using an exemplary algal species, two exemplary aerobic bacterial species, and two exemplary diazotrophs (e.g., diazotrophic bacteria).

Specifically, a production culture for continuous and symbiotic algal growth was established by inoculating surface water with algal species Chlorella vulgaris, Chlorella sp. D101, two aerobic bacterial species Rhodobacter sphaeroides, Rhodobacter sp. D788 and Bacillus sp D320, and two diazotrophic bacterial species Spirulina maxima, Spirulina sp. D11 and Methanobacteria sp D422. All parameters in this Example were identical to those disclosed in Example 2, except that an additional diazotroph, Methanobacteria sp D422, was added to the co-culture. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively.

### Results

The results from this experiment showed that the algal yields were comparable to the yields from Example 2 (e.g., 0.45 g/L/day) of dry weight algae. In addition, the results indicated that addition of the Methanobacteria sp D422, was compatible with very long-term sustained symbiotic co-culture.

As of the filing date of this Application, the continuous, symbiotic co-culture of this Example with an additional diazotroph is ongoing and has been maintained for 3 years.

### Example 4

### Continuous, Symbiotic Co-Cultures of Scenedesmus obliquus, Scenedesmus sp. D202, Bacillus sp D320, Rhodobacter sphaeroides, Rhodobacter sp. D788, Methanobacteria sp D422, and Spirulina maxima, Spirulina sp. D11 were Established and Maintained for Continuous Sustained Symbiotic Growth

Example Overview. In this working Example 4, a continuous, symbiotic sustainable co-culture was established in a suitable culture vessel using an exemplary algal species, two exemplary aerobic bacterial species, and two exemplary diazotrophs (e.g., diazotrophic bacteria).

Specifically, a production culture vessel for continuous and symbiotic growth was established by inoculating with algal species Scenedesmus obliquus, Scenedesmus sp. D202, two aerobic bacterial species Bacillus sp D320 and Rhodobacter sphaeroides, Rhodobacter sp. D788, and two diazotrophic bacterial species Methanobacteria sp D422 and Spirulina maxima, Spirulina sp. D11. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively. The production culture vessel was an open raceway concrete vessel container having the dimensions of 6×40 m². Growth medium (surface water) was added via inlet valve to the vessel to a depth of 1 m and circulated via paddle wheel. Throughout the experiment, the depth of the growth medium was kept constant at 1 m by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1. The remaining parameters were identical to those disclosed in Example 2.

Periodically, throughout the growth cycle Scenedesmus obliquus, Scenedesmus sp. D202 was harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10%, 15%, 20%, 25%, 30%, 35%, 40%, 45%, or 50%) of algae in the culture was harvested such that the algae remaining after harvest is sufficient to maintain (e.g., re-establish and/or sustain) the symbiotic co-culture. As of the filing date of this Application, the continuous, symbiotic co-culture of this Example has been maintained for 3.5 years.

### Results

In this Example, Applicant discovered a high yield of dry weight Scenedesmus obliquus, Scenedesmus sp. D202 (e.g., 0.40 g/L/day) was sustainable, by establishing a symbiotic co-culture of an aerobic bacteria and a diazotroph with the algae as disclosed herein. See Example 6.

### Example 5

### Continuous, Symbiotic Co-Cultures of Euglena gracilis, Euglena sp. D405, Bacillus sp D320, Rhodobacter sphaeroides, Rhodobacter sp. D788, Methanobacteria sp D422, and Spirulina maxima, Spirulina sp. D11 were Established and Maintained for Continuous Symbiotic Growth

Example Overview. In this working Example 5, a continuous, symbiotic sustainable co-culture was established in a suitable culture vessel using an exemplary algal species, two exemplary aerobic bacterial species, and two exemplary diazotrophs (e.g., diazotrophic bacteria).

Specifically, a production culture vessel for continuous and symbiotic growth was established by inoculating with algal species Euglena gracilis, Euglena sp. D405, the two aerobic bacterial species Bacillus sp D320 and Rhodobacter sphaeroides, Rhodobacter sp. D788, and two diazotrophic bacterial species Methanobacteria sp D422 and Spirulina maxima, Spirulina sp. D11. Typically, in this Example, the relative amounts of the algal including cyanobacteria, aerobic bacterial and diazotrophic organisms (bacteria and archaea excluding cyanobacteria) were maintained in a ratio or proportion of about 100:1.6:0.18, respectively. The production culture vessel comprised an open concrete vessel container having the dimensions of 25×1500 m². Growth medium was added from a tap through sand filter to the vessel to a depth of 1.2 m and circulated using a paddle wheel-type device. Throughout the experiment, the depth of the growth medium was kept constant at 1.2 m by manual addition or automatically with a float ball, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1. The remaining parameters were identical to those disclosed in Example 2.

Periodically, throughout the growth cycle Euglena gracilis, Euglena sp. D405 was harvested from the growth medium by skimming the top of the culture and/or collecting from the bottom or from the bulk of the vessel via pumping and filtering. The frequency and extent of algal harvesting was suitable to provide for sustained symbiotic co-culture of the algal, aerobic bacterial and diazotrophic organisms. Typically, only a fraction (e.g., 10%, 15%, 20%, 25%, 30%, 35%, 40%, 45%, or 50%)) of algae in the culture was harvested such that the algae remaining after harvest is sufficient to re-establish and/or sustain the symbiotic co-culture. As of the filing date of this Application, the continuous, symbiotic co-culture of this Example was maintained for 3.5 years.

### Results

In this Example, a high yield of dry weight Euglena gracilis, Euglena sp. D405 (e.g., 0.39 g/L/day) was sustainable, by establishing a symbiotic co-culture of an aerobic bacteria and a diazotroph with the algae as disclosed herein. See Example 6.

### Example 6

### Growth of Chlorella vulgaris, Chlorella sp. D101 Cultured Solo in Low Nutrient Medium Compared to the Growth of Chlorella sp. Chlorella vulgaris, Chlorella sp. D101 Cultured in Combination with Bacillus sp D320 and Two Diazotrophs, Methanobacteria sp D422, and Spirulina maxima, Spirulina sp. D11, in Low Nutrient Medium

Example Overview. In this working Example 6, the growth of a culture of only an exemplary algal species was compared to the growth of an exemplary algal species co-cultured with an exemplary aerobic bacterial species, and two exemplary diazotrophs where both the solo culture and the co-culture are in low nutrient medium.

Specifically, a culture vessel was established by inoculating surface water with either algal species Chlorella vulgaris, Chlorella sp. D101 alone or algal species Chlorella vulgaris, Chlorella sp. D101 co-cultured with an aerobic bacterial species Bacillus sp D320, and two diazotrophic bacterial species, Methanobacteria sp D422, and Spirulina maxima, Spirulina sp. D11. The culture vessels were rectangular open plastic containers having the dimensions of 1.25×2.75 m². Either high nutrient culture medium (as described under Example 1) or surface water was added via batch flow to the vessel to a depth of 40 cm and circulated by using a pump. One culture of Chlorella vulgaris, Chlorella sp. D101 alone was grown in the high nutrient culture medium. The remaining culture of Chlorella vulgaris, Chlorella sp. D101 alone was incubated in surface water. The co-culture of Chlorella vulgaris, Chlorella sp. D101, Bacillus sp D320, Methanobacteria sp D422, and Spirulina maxima, Spirulina sp. D11 was grown in surface water. Throughout the experiment the depth of the growth media or surface water was kept constant at 40 cm by manual addition, as described under Example 1. Natural sunlight was used and was continuously cycled in alternating periods of approximately 12 hours of light and 12 hours of darkness. The temperature was maintained between 25-30° C. by cold water circulation or heating by exchanging heat with waste steam as described under Example 1. The remaining parameters were identical to those disclosed in Example 2.

### Results

In this Example, Applicant discovered, base on her yield data as shown in Table 6, that the only consistent way to get algae alone to grow and give reasonable yields (e.g., 0.45 g/L/day) is to use the high nutrient culture medium, which has substantial amounts of exogenous chemical fertilizers/nutrients. Without the substantial amount of exogenous chemical fertilizers/nutrients (e.g., as in when only surface water is used as the medium), there was no consistent yield (e.g., 0.0065 g/L/day; i.e., 1.4% compared to yield using exogenous chemical fertilizers/nutrients). However, when Applicant sustained symbiotic co-culture methods (e.g., growing at least one algal strain with at least one aerobic bacteria and at least one diazotroph) were used with only surface water as the growth medium, they obtained algae yields comparable to the algae yields obtained by growing algae using the high nutrient culture medium containing exogenous chemical fertilizers/nutrients.

While this is an exact experiment with specific yields of algae, the range of yields can be 0.05 g/L/day to 2.5 g/L/day (dry weight) of algae. In certain embodiments, the yield depends upon the growth conditions, which varies according to many different circumstances, including but not limited to weather (e.g., temperature, precipitation, and/or evaporation), pH, and source of surface water.

TABLE 6
Comparison between the growth of Chlorella vulgaris, Chlorella sp. D101 grown
alone in high and low nutrient and Chlorella vulgaris, Chlorella sp. D101 grown
in combination with aerobic bacterial species Bacillus sp D320, and two diazotrophic
bacterial species Methanobacteria sp D422, and Spirulina maxima, Spirulina sp. D11.

| | Chlorella | Chlorella | Chlorella vulgaris, Chlorella |
|---|---|---|---|
| | vulgaris, | vulgaris, | sp. D101 + Bacillus sp D320 + |
| | Chlorella sp. | Chlorella sp. | Methanobacteria sp D422 + |
| Culture | D101 (alone) | D101 (alone) | Spirulina maxima, Spirulina sp. D11 |
| Type of culture | High yield culture | Surface water | Surface water |
| medium used to grow | medium | | |
| the algae | | | |
| Average yield per day | 0.45 g/L | 0.0065 g/L | 0.42 g/L |
| (dry weight) | | | |

### Example 7

### In Preferred Embodiments, the Inventive Symbiotic Co-Cultures Comprising a Diazotroph Provide for Enhanced Oil Production on a Per-Algal Cell Basis

As recognized in the art (e.g., Hu, et al., The Plant Journal, 54:621-639, 2008; Alonso et al., Phytochemistry 54:461-471, 2000; Renaud et al., Aquaculture 211:195-214, 2002, all incorporated herein by reference, and in particular for their teachings one oil content and lipid and fatty acid compositions), stress of algae, and particularly based on nitrogen deprivation enhances (e.g., on a per cell basis) oil production by the stressed algae. According to additional aspects, the inventive symbiotic co-cultures comprising a diazotroph provide for enhanced oil production (e.g., sustained enhanced oil production) by the algae compared to oil production by non-nitrogen-stressed algae in cultures lacking a diazotroph. Without being bound by mechanism, the disclosed inventive use of a diazotroph in the inventive symbiotic co-cultures, in the absence of exogenously added chemical nitrogen, or under nitrogen stress conditions, not only provides bioavailable nitrogen, but also unexpectedly provides for enhanced oil production by the algal component (e.g., on a per cell basis) of the symbiotic co-culture by providing an amount of bio-available nitrogen that is, on the one hand, sufficient to provide for healthy algal growth within the co-culture without, on the other hand, abrogating the art-recognized nitrogen-stress-mediated enhancement of oil production by the algae.

Therefore, according to preferred aspects, maintaining a balanced symbiotic co-culture as described herein not only enables algal growth using low exogenous nutrient growth addition, but enables algal growth with an enhanced oil yield (e.g., on a per-cell basis) using low exogenous nutrient growth addition. Applicant refers to this as continuous symbiotic diazotroph-attenuated nitrogen stress co-cultivation (DANSC).

While nitrogen stress responses in algae are known in the art, prior art attempts at using nitrogen stress to induce algal bioproduct production have been limited to closed-system bioreactors where algae are initially non-symbiotically grown in rich medium to provide a large algal biomass, followed by imposing nitrogen deprivation by exhaustion and/or adjustment of nutrients in the medium of the closed system to induce nitrogen stress responses, followed by harvesting of the complete nitrogen stressed algal biomass; that is, prior art methods comprise non-continuous batch processes that are suitable for closed systems only. By contrast, Applicant's inventive methods comprise the use of symbiotic diazotroph-attenuated nitrogen stress co-cultivation (DANSC), as disclosed and taught herein, to provide for a continuous symbiotic co-culture using diazotroph-attenuated nitrogen stress such that the advantages of nitrogen stress for algal bioproduct production can be implemented and sustained continuously in batch or non-batch processes, and in open and/or closed cultivation systems.

As appreciated in the art, most algae grown alone under non-stressed conditions (not symbiotically co-cultured as disclosed herein) typically have a total lipid content of about 25 to about 27% DCW (% of dry cell weight), predominantly of saturated and monounsaturated fatty acids (e.g., C14-C18) (e.g., C16:0, C16:1, C18:1, C20:1, etc., depending on the species), and some polyunsaturated fatty acids (PUFAs) (e.g., C18:2, C18:3ω3, C18:5ω3, etc., depending on the species) (see, e.g., Hu, et al., The Plant Journal, 54:621-639, 2008; incorporated herein by reference, and in particular for its teachings one oil content and lipid and fatty acid compositions on pages 623-625 and Table 1 on page 625). Moreover, in non-stressed cells, a high proportion of the fatty acids are in the form of membrane phospholipids, etc., with some in the form of neutral lipids (neutral triacylglycerols (TAGs)). By contrast, under stress, conditions, total lipids are known to increase, and the increase is primarily in the accumulation of neutral triacylglycerols (TAGs), which may account for as much as 80% of the total lipid in stressed cells, due to both de novo biosynthesis and conversion of existing membrane lipids into TAGs.

According to additional aspects, therefore, maintaining a balanced symbiotic co-culture as described herein not only enables algal growth using low exogenous nutrient growth addition, but surprisingly enables algal growth with an enhanced oil yield (e.g., on a per-cell basis) using low exogenous nutrient growth addition, and further enables accumulation of higher percentage of TAGs, which are a preferred starting material for biodiesel production by transesterification of TAGs (e.g., with methanol), and further enables modulation of the structure and extent of saturation of the fatty acid components. Moreover, according to additional aspects, because the key properties (ignition quality (cetane number), cold-flow properties and oxidative stability) of biodiesel are largely determined by the structure and extent of unsaturation of its component fatty acids esters, the inventive symbiotic co-culture methods provide for production of superior biofuels. Saturated fats produce biodiesel having superior oxidative stability and higher cetane number, but poor low-temperature properties (e.g., gelling at low temperatures), whereas PUFAs produce biodiesel having good cold-flow properties, but are susceptible to oxidation. Therefore, the balance of unsaturation and saturation is an important aspect of the quality and properties of biofuels derived from TAGs.

According to particular aspects of the present invention, maintaining a balanced symbiotic co-culture as described herein not only enables algal growth using low exogenous nutrient growth addition, but surprisingly enables algal growth with an enhanced oil yield (e.g., on a per-cell basis) using low exogenous nutrient growth addition, additionally enables accumulation of higher percentage of TAGs, and further enables accumulation of TAGs that provide for an optimal balance of unsaturated and saturated fatty acid esters, and hence in the key properties (ignition quality (cetane number), cold-flow properties and oxidative stability) of biofuels derived therefrom.

According to particular aspects, maintaining a balanced symbiotic co-culture as described herein surprisingly enables algal growth, using low exogenous nutrient growth addition, with an enhanced oil yield (e.g., on a per-cell basis), and wherein the total lipid content is enhanced to a level equal to or greater than about: 30%, 35%, 40%, 45%, or 50% DCW, or enhanced to a value in the range of from about 30% to about 50% DCW.

According to additional aspects, maintaining a balanced symbiotic co-culture as described herein surprisingly enables algal growth, using low exogenous nutrient growth addition, with an enhanced oil yield (e.g., on a per-cell basis), wherein the amount of total lipid in the form of TAGs is equal to or greater than about: 20%, 30%, 40%, 50%, 60%, 70% or 80% DCW of the total lipid.

According to further aspects, maintaining a balanced symbiotic co-culture as described herein surprisingly enables algal growth, using low exogenous nutrient growth addition, with an enhanced oil yield (e.g., on a per-cell basis) comprising an increased percentage (relative to PUFAs) of saturated and mono-saturated fatty acids in the TAGs, thereby providing an oil product having a TAG composition and structure that provides more optimal balance of key properties of ignition quality (cetane number), cold-flow properties and oxidative stability for any biofuels derived therefrom.

1. A method for enhanced sustainable production of algal bioproducts, comprising:
providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel;
inoculating the cultivation medium in the vessel with at least one algal species, at least one aerobic bacterial species and at least one diazotroph;
continuously cultivating the inocula under sustainable symbiotic co-culture conditions to provide for diazotroph-assisted sustained production of a harvestable amount of algal biomass; and
repetitive harvesting of a portion of the algal biomass from the continuous co-culture, to provide for enhanced sustainable production of at least one algal bioproduct.

2. The method of claim 1, wherein at least a portion of the algal growth in the co-culture is photosynthetic.

3. The method of claim 1, wherein algal growth comprises both heterotrophic and autotrophic growth.

4. The method of claim 1, wherein inoculating comprises use of an initial inoculum ratio of algae:aerobic bacteria:diazotroph selected from the group consisting of: 100:1.6:0.18; 10:1.6:18; 50-500:0.8-80:0.09-9; and 10-1000:0.16-160:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:1.6:0.18; 100:25:18; 50-500:0.8-80:0.09-9; and 10-1000:0.16-160:0.018-18, or comprises a sustained symbiotic ratio of algae:aerobic bacteria:diazotroph, including dead biomass, selected from the group consisting of: 110:10:1.5; 150:50:15; 55-550:5-50:0.75-7.5; and 15-1100:1-100:0.15-15.

5. The method of claim 1, further comprising:
monitoring the at least one of CO₂, O₂, nitrogen, and pH levels in the cultivation medium; and
adjusting the at least one of CO₂, O₂, nitrogen, and pH levels in the cultivation medium as required to provide for sustainable symbiotic co-culture of the at least one algal species, the at least one aerobic bacterial species and the at least one diazotroph.

6. The method of claim 1, further comprising isolating at least one algal bioproduct from the harvested algal biomass.

7. The method of claim 1, wherein sustainable growth of the at least one algal species, the at least one aerobic bacterial species and the at least one diazotroph, is maintained with low nutrient addition.

8. The method of claim 1, comprising use of minimal addition of exogenous nutrients, and wherein at least 5% of the macronutrient driving growth in the symbiotic co-culture derive from decomposed algal and bacterial cells produced during the co-cultivating.

9. The method of claim 1, wherein the aqueous cultivation medium comprises at least one of ground water, surface water, brackish water, salt water, sea water, marine water, lake water, river water, waste water, and tap water.

10. The method of claim 1, wherein the cultivation medium is suitable to induce at least one nitrogen stress response in the algal cells cultured therein.

11. The method of claim 10, wherein the diazotroph component is maintained in an amount sufficient to sustainably attenuate the at least one nitrogen stress response in the symbiotically co-cultivated algal cells.

12. The method of claim 1, wherein at least a portion of the CO₂ present in the cultivation medium is endogenously derived from the aerobic bacterial component of the co-culture, wherein at least a portion of the nitrogen present in the cultivation medium is endogenously derived from the diazotrophic component of the co-culture, and wherein at least a portion of the O₂ present in the cultivation medium is endogenously derived from the algal component of the co-culture.

13. The method of any one of claims 1, 10 and 11, wherein the co-culture provides, on a per-algal cell basis, relative to non-symbiotic growth of the respective algal cells, for at least one of: enhanced total lipid production; enhanced production of triacylglycerols (TAGs); enhanced percentage of total lipid as TAGs; and enhanced percentage of saturated and mono-saturated fatty acids, relative to polyunsaturated fatty acids (PUFAs), in TAGs.

14. The method of claim 13, wherein the total lipid content is enhanced to a level equal to or greater than: 30%; 35%; 40%; 45%; or 50% dry cell weight (DCW), or enhanced to a value in the range of from about 30% to about 50% DCW.

15. The method of claim 13, wherein the amount of total lipid in the form of triacylglycerols (TAGs) is equal to or greater than: 20%; 30%; 40%; 50%; 60%; 70%; or 80% dry cell weight (DCW) of the total lipid, or in the range of from about 30% to about 80% DCW of the total lipid.

16. The method of claim 13, wherein the increased percentage, relative to polyunsaturated fatty acids (PUFAs), of the saturated and mono-saturated fatty acids in the triacylglycerols (TAGs), is at least: 5%; 10%; 20%; 30% dry cell weight (DCW); or greater, or is in the range of from about 10% to about 30% DCW.

17. The method of claim 1, wherein the at least one diazotroph is selective from the diazotrophic bacterial group consisting of photosynthetic, non-photosynthetic, anaerobic, aerobic, methanogenic, sulfurgenic, symbiotic diazotrophes, cyanobacteria, and oxygenic and anoxygenic forms thereof.

18. The method of claim 1, wherein the at least one algal species, at least one aerobic bacterial species and at least one diazotroph comprises at least one organism according to Tables 1-4 as disclosed herein.

19. A method for enhanced sustainable production of algal bioproducts, comprising:
providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel, the cultivation medium suitable to induce at least one nitrogen stress response in algal cells cultured therein;
inoculating the cultivation medium in the vessel with at least one algal species, at least one aerobic bacterial species and at least one diazotroph;
continuously cultivating the inocula under sustainable symbiotic co-culture conditions, wherein the diazotroph component is maintained in an amount sufficient to sustainably attenuate the at least one nitrogen stress response in the symbiotically co-cultivated algal cells to provide for diazotroph-assisted sustained production of a harvestable amount of algal biomass; and
repetitive harvesting of a portion of the algal biomass from the continuous co-culture, to provide for enhanced sustainable production of at least one algal bioproduct.

20. The method of claim 19, wherein at least a portion of the algal growth in the co-culture is photosynthetic.

21. The method of claim 19, wherein algal growth comprises both heterotrophic and autotrophic growth.

22. The method of claim 19, wherein inoculating comprises use of an initial inoculum ratio of algae:aerobic bacteria:diazotroph selected from the group consisting of: 100:1.6:0.18; 10:1.6:18; 50-500:0.8-80:0.09-9; and 10-1000:0.16-160:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:1.6:0.18; 100:25:18; 50-500:0.8-80:0.09-9; and 10-1000:0.16-160:0.018-18, or comprises a sustained symbiotic ratio of algae:aerobic bacteria:diazotroph, including dead biomass, selected from the group consisting of: 110:10:1.5; 150:50:15; 55-550:5-50:0.75-7.5; and 15-1100:1-100:0.15-15.

23. The method of claim 19, further comprising:
monitoring the at least one of CO₂, O₂, nitrogen, and pH levels in the cultivation medium; and
adjusting the at least one of CO₂, O₂, nitrogen, and pH levels in the cultivation medium as required to provide for sustainable symbiotic co-culture of the at least one algal species, the at least one aerobic bacterial species and the at least one diazotroph.

24. The method of claim 19, further comprising isolating at least one algal bioproduct from the harvested algal biomass.

25. The method of claim 19, wherein sustainable growth of the at least one algal species, the at least one aerobic bacterial species and the at least one diazotroph, is maintained with low nutrient addition.

26. The method of claim 19, comprising use of minimal addition of exogenous nutrients, and wherein at least 5% of the macronutrient driving growth in the symbiotic co-culture derive from decomposed algal and bacterial cells produced during the co-cultivating.

27. The method of claim 19, wherein the aqueous cultivation medium comprises at least one of ground water, surface water, brackish water, salt water, sea water, marine water, lake water, river water, waste water, and tap water.

28. The method of claim 19, wherein at least a portion of the CO₂ present in the cultivation medium is endogenously derived from the aerobic bacterial component of the co-culture, wherein at least a portion of the nitrogen present in the cultivation medium is endogenously derived from the diazotrophic component of the co-culture, and wherein at least a portion of the O₂ present in the cultivation medium is endogenously derived from the algal component of the co-culture.

29. The method of claim 19, wherein the co-culture provides, on a per-algal cell basis, relative to non-symbiotic growth of the respective algal cells, for at least one of: enhanced total lipid production; enhanced production of triacylglycerols (TAGs); enhanced percentage of total lipid as TAGs; and enhanced percentage of saturated and mono-saturated fatty acids, relative to polyunsaturated fatty acids (PUFAs), in TAGs.

30. The method of claim 29, wherein the total lipid content is enhanced to a level equal to or greater than: 30%; 35%; 40%; 45%; or 50% dry cell weight (DCW), or enhanced to a value in the range of from about 30% to about 50% DCW.

31. The method of claim 29, wherein the amount of total lipid in the form of triacylglycerols (TAGs) is equal to or greater than: 20%; 30%; 40%; 50%; 60%; 70%; or 80% dry cell weight (DCW) of the total lipid, or in the range of from about 30% to about 80% DCW of the total lipid.

32. The method of claim 29, wherein the increased percentage, relative to polyunsaturated fatty acids (PUFAs), of the saturated and mono-saturated fatty acids in the triacylglycerols (TAGs), is at least: 5%; 10%; 20%; 30% dry cell weight (DCW); or greater, or is in the range of from about 10% to about 30% DCW.

33. The method of claim 19, wherein the at least one diazotroph is selective from the diazotrophic bacterial group consisting of photosynthetic, non-photosynthetic, anaerobic, aerobic, methanogenic, sulfurgenic, symbiotic diazotrophes, cyanobacteria, and oxygenic and anoxygenic forms thereof.

34. The method of claim 19, wherein the at least one algal species, at least one aerobic bacterial species and at least one diazotroph comprises at least one organism according to Tables 1-4 as disclosed herein.

35. A method for enhanced sustainable production of algal bioproducts, comprising:
providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel, the cultivation medium suitable to induce at least one nitrogen stress response in algal cells cultured therein;
inoculating the cultivation medium in the vessel with at least one algal species, at least one aerobic bacterial species and at least one diazotroph;
continuously cultivating the inocula under sustainable symbiotic co-culture conditions, wherein at least a portion of the algal growth in the co-culture is photosynthetic, and wherein the diazotroph component is maintained in an amount sufficient to sustainably attenuate the at least one nitrogen stress response in the symbiotically co-cultivated algal cells to provide for diazotroph-assisted sustained production of a harvestable amount of algal biomass; and
repetitive harvesting of a portion of the algal biomass from the continuous co-culture, to provide for enhanced sustainable production of at least one algal bioproduct.

36. The method of claim 35, wherein algal growth comprises both heterotrophic and autotrophic growth.

37. The method of claim 35, wherein inoculating comprises use of an initial inoculum ratio of algae:aerobic bacteria:diazotroph selected from the group consisting of: 100:1.6:0.18; 10:1.6:18; 50-500:0.8-80:0.09-9; and 10-1000:0.16-160:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:1.6:0.18; 100:25:18; 50-500:0.8-80:0.09-9; and 10-1000:0.16-160:0.018-18, or comprises a sustained symbiotic ratio of algae:aerobic bacteria:diazotroph, including dead biomass, selected from the group consisting of: 110:10:1.5; 150:50:15; 55-550:5-50:0.75-7.5; and 15-1100:1-100:0.15-15.

38. The method of claim 35, further comprising:
monitoring the at least one of CO₂, O₂, nitrogen, and pH levels in the cultivation medium; and
adjusting the at least one of CO₂, O₂, nitrogen, and pH levels in the cultivation medium as required to provide for sustainable symbiotic co-culture of the at least one algal species, the at least one aerobic bacterial species and the at least one diazotroph.

39. The method of claim 35, further comprising isolating at least one algal bioproduct from the harvested algal biomass.

40. The method of claim 35, wherein sustainable growth of the at least one algal species, the at least one aerobic bacterial species and the at least one diazotroph, is maintained with low nutrient addition.

41. The method of claim 35, comprising use of minimal addition of exogenous nutrients, and wherein at least 5% of the macronutrient driving growth in the symbiotic co-culture derive from decomposed algal and bacterial cells produced during the co-cultivating.

42. The method of claim 35, wherein the aqueous cultivation medium comprises at least one of ground water, surface water, brackish water, salt water, sea water, marine water, lake water, river water, waste water, and tap water.

43. The method of claim 35, wherein at least a portion of the CO₂ present in the cultivation medium is endogenously derived from the aerobic bacterial component of the co-culture, wherein at least a portion of the nitrogen present in the cultivation medium is endogenously derived from the diazotrophic component of the co-culture, and wherein at least a portion of the O₂ present in the cultivation medium is endogenously derived from the algal component of the co-culture.

44. The method of claim 35, wherein the co-culture provides, on a per-algal cell basis, relative to non-symbiotic growth of the respective algal cells, for at least one of: enhanced total lipid production; enhanced production of triacylglycerols (TAGs); enhanced percentage of total lipid as TAGs; and enhanced percentage of saturated and mono-saturated fatty acids, relative to polyunsaturated fatty acids (PUFAs), in TAGs.

45. The method of claim 44, wherein the total lipid content is enhanced to a level equal to or greater than: 30%; 35%; 40%; 45%; or 50% dry cell weight (DCW), or enhanced to a value in the range of from about 30% to about 50% DCW.

46. The method of claim 44, wherein the amount of total lipid in the form of triacylglycerols (TAGs) is equal to or greater than: 20%; 30%; 40%; 50%; 60%; 70%; or 80% dry cell weight (DCW) of the total lipid, or in the range of from about 30% to about 80% DCW of the total lipid.

47. The method of claim 44, wherein the increased percentage, relative to polyunsaturated fatty acids (PUFAs), of the saturated and mono-saturated fatty acids in the triacylglycerols (TAGs), is at least: 5%; 10%; 20%; 30% dry cell weight (DCW); or greater, or is in the range of from about 10% to about 30% DCW.

48. The method of claim 35, wherein the at least one diazotroph is selective from the diazotrophic bacterial group consisting of photosynthetic, non-photosynthetic, anaerobic, aerobic, methanogenic, sulfurgenic, symbiotic diazotrophes, cyanobacteria, and oxygenic and anoxygenic forms thereof.

49. The method of claim 35, wherein the at least one algal species, at least one aerobic bacterial species and at least one diazotroph comprises at least one organism according to Tables 1-4 as disclosed herein.

50. A method for enhanced sustainable production of algal bioproducts, comprising:
providing a cultivation vessel containing an aqueous cultivation medium therein, the cultivation vessel in operative communication with suitable detection means to measure at least one of CO₂, O₂, nitrogen, and pH levels in the cultivation medium, and having an inlet in operative communication with a source of cultivation medium, and an outlet operative with the inlet and the cultivation vessel to provide for exchange of cultivation medium within the vessel;
inoculating the cultivation medium in the vessel with at least one algal species, and at least one diazotroph;
continuously cultivating the inocula under sustainable symbiotic co-culture conditions to provide for diazotroph-assisted sustained production of a harvestable amount of algal biomass; and
repetitive harvesting of a portion of the algal biomass from the continuous co-culture, to provide for enhanced sustainable production of at least one algal bioproduct.

51. The method of claim 50, wherein at least a portion of the algal growth in the co-culture is photosynthetic.

52. The method of claim 50, wherein algal growth comprises both heterotrophic and autotrophic growth.

53. The method of claim 50, wherein inoculating comprises use of an initial inoculum ratio of algae:diazotroph selected from the group consisting of: 100:0.18; 10:18; 50-500:0.09-9; and 10-1000:0.018-18, and/or wherein continuously cultivating comprises at least periodically monitoring the organismal ratios and adjusting same as required to maintain a sustained symbiotic ratio of algae:aerobic bacteria:diazotroph, excluding dead biomass, selected from the group consisting of: 100:0.18; 100:18; 50-500:0.09-9; and 10-1000:0.018-18, or comprises a sustained symbiotic ratio of algae:aerobic bacteria:diazotroph, including dead biomass, selected from the group consisting of: 110:1.5; 150:15; 55-550:0.75-7.5; and 15-1100:0.15-15.

54. The method of claim 50, further comprising:
monitoring the at least one of CO₂, O₂, nitrogen, and pH levels in the cultivation medium; and
adjusting the at least one of CO₂, O₂, nitrogen, and pH levels in the cultivation medium as required to provide for sustainable symbiotic co-culture of the at least one algal species and the at least one diazotroph.

55. The method of claim 50, further comprising isolating at least one algal bioproduct from the harvested algal biomass.

56. The method of claim 50, wherein sustainable growth of the at least one algal species and the at least one diazotroph is maintained with low nutrient addition.

57. The method of claim 50, comprising use of minimal addition of exogenous nutrients, and wherein at least 5% of the macronutrient driving growth in the symbiotic co-culture derive from decomposed algal and diazotroph cells produced during the co-cultivating.

58. The method of claim 50, wherein the aqueous cultivation medium comprises at least one of ground water, surface water, brackish water, salt water, sea water, marine water, lake water, river water, waste water, and tap water.

59. The method of claim 50, wherein the cultivation medium is suitable to induce at least one nitrogen stress response in the algal cells cultured therein.

60. The method of claim 59, wherein the diazotroph component is maintained in an amount sufficient to sustainably attenuate the at least one nitrogen stress response in the symbiotically co-cultivated algal cells.

61. The method of claim 50, wherein at least a portion of the nitrogen present in the cultivation medium is endogenously derived from the diazotrophic component of the co-culture, and wherein at least a portion of the O₂ present in the cultivation medium is endogenously derived from the algal component of the co-culture.

62. The method of any one of claims 59, 59 and 60, wherein the co-culture provides, on a per-algal cell basis, relative to non-symbiotic growth of the respective algal cells, for at least one of: enhanced total lipid production; enhanced production of triacylglycerols (TAGs); enhanced percentage of total lipid as TAGs; and enhanced percentage of saturated and mono-saturated fatty acids, relative to polyunsaturated fatty acids (PUFAs), in TAGs.

63. The method of claim 62, wherein the total lipid content is enhanced to a level equal to or greater than: 30%; 35%; 40%; 45%; or 50% dry cell weight (DCW), or enhanced to a value in the range of from about 30% to about 50% DCW.

64. The method of claim 62, wherein the amount of total lipid in the form of triacylglycerols (TAGs) is equal to or greater than: 20%; 30%; 40%; 50%; 60%; 70%; or 80% dry cell weight (DCW) of the total lipid, or in the range of from about 30% to about 80% DCW of the total lipid.

65. The method of claim 62, wherein the increased percentage, relative to polyunsaturated fatty acids (PUFAs), of the saturated and mono-saturated fatty acids in the triacylglycerols (TAGs), is at least: 5%; 10%; 20%; 30% dry cell weight (DCW); or greater, or is in the range of from about 10% to about 30% DCW.

66. The method of claim 50, wherein the at least one diazotroph is selective from the diazotrophic bacterial group consisting of photosynthetic, non-photosynthetic, anaerobic, aerobic, methanogenic, sulfurgenic, symbiotic diazotrophes, cyanobacteria, and oxygenic and anoxygenic forms thereof.

67. The method of claim 50, wherein the at least one algal species and the at least one diazotroph comprises at least one organism according to Tables 1-4 as disclosed herein.
